(19) United States
(12) Patent Application Publication
(10) Pub. No.: US 2026/0356795 A1
(43) Pub. Date: Nov. 12, 2026

(54) DIFLUOROMETHYLENYL-CONTAINING AMINOCYCLOPENTANECARBOXYLIC ACID ENZYME INACTIVATORS

(71) Applicant: Northwestern University, Evanston, IL (US)

(72) Inventors: Richard Bruce Silverman, Evanston, IL (US); Koon Mook Kang, Evanston, IL (US)

(21) Appl. No.: 19/660,961

(22) Filed: Apr. 28, 2026

Related U.S. Application Data
(60) Provisional application No. 63/796,198, filed on Apr. 28, 2025.

(51) Int. Cl. C07C 229/48; A61K 31/196; A61P 25/00

(52) U.S. Cl. CPC C07C 229/48; A61K 31/196; A61P 25/00

(57) ABSTRACT

Disclosed herein are compounds and compositions as selective γ-aminobutyric acid aminotransferase (GABA-AT) inactivators. Also disclosed herein are methods of using the same, such as methods of treating a disease or disorder associated with GABA-AT activity, including a neurological disease or disorder, pain, and a cancer.

### CROSS-REFERENCES TO RELATED APPLICATIONS

The present patent application claims priority from U.S. Provisional patent application No. 63/796,198, filed on Apr. 28, 2025, the entirety of each such application is hereby incorporated by reference herein as if fully set forth herein.

### STATEMENT REGARDING FEDERALLY SPONSORED RESEARCH

This invention was made with government support under grant number NS123057 awarded by the National Institutes of Health. The government has certain rights in the invention.

### BACKGROUND

Since being discovered in the mammalian brain, γ-aminobutyric acid (GABA) has been regarded as the main inhibitory neurotransmitter¹. Dysfunction in GABA regulation has been reported to correlate with various neurological conditions, such as epilepsy² and age-related cognitive decline³. GABA aminotransferase (GABA-AT), a pyridoxal phosphate (PLP)-dependent enzyme, converts GABA to succinic semialdehyde by transferring its γ-amino group⁴ (FIG. 1A and Scheme 1). Therefore, preventing GABA degradation by inactivating GABA-AT can be a useful alternative strategy to raise its level in the central nervous system (CNS)⁵, rather than administering GABA itself, which is unable to permeate the brain-blood barrier (BBB). Vigabatrin (γ-vinyl GABA, 4-amino-5-hexenoic acid, FIG. 2) is the only FDA-approved mechanism-based inactivator of GABA-AT⁶ and thus has been used as a first-line therapy for infantile spasms⁷. Vigabatrin inactivates GABA-AT by acting as its substrate. It forms the external aldimine intermediate with PLP followed by tautomerization, then mainly (70%) undergoes Michael addition to its γ-vinyl group by the catalytic lysine (Lys329)⁸ (Scheme 2) In addition to the Michael addition mechanism, vigabatrin inactivates GABA-AT by forming an enamine-driven ternary adduct via an enamine intermediate (Scheme 2).

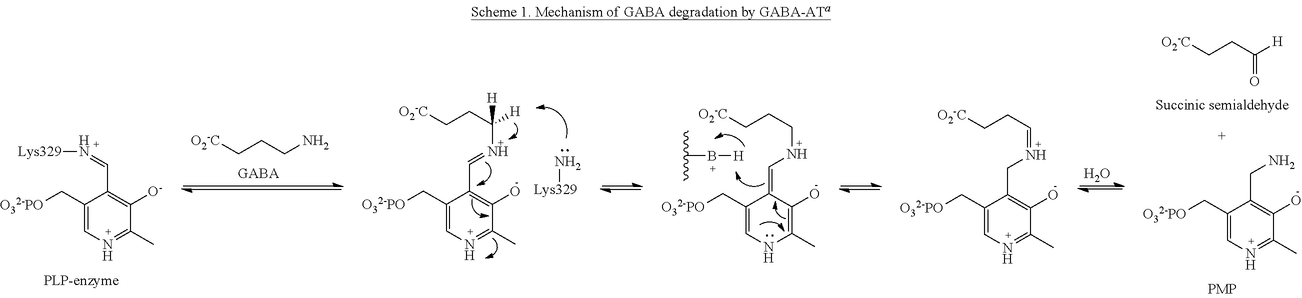

^{a}GABA, γ-aminobutyric acid; PLP, pyridoxal-5-phosphate; PMP, pyridoxamine-5-phosphate.

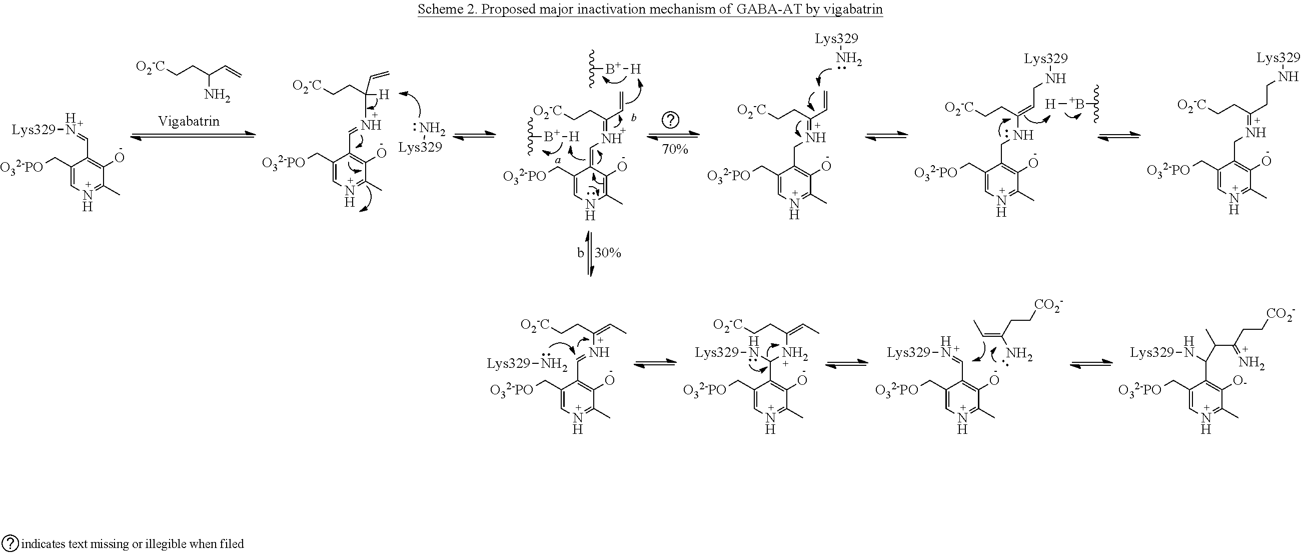

We previously have reported the design, synthesis, and in vitro evaluation of a new highly effective mechanism-based inactivator, CPP-115⁹ (k_{inact}/K_{I}=34.9 min⁻¹mM⁻¹). Additionally, an effort to enhance this inactivator led to the development of a 9.8 times more efficient inactivator, OV329⁶ (k_{inact}/K_{I}=342 min⁻¹mM⁻¹). Both mechanism-based inactivators have been recognized to inactivate GABA-AT by similar mechanisms, considering their time-dependent inactivation and structural similarity⁶. The potential inactivation mechanism of CPP-115 has been proposed by the results from its fluoride ion release assay, mass spectrometric analysis, and X-ray crystallography¹⁰. Upon binding, CPP-115 initiates a rapid formation of the corresponding external aldimine intermediate (1), attacking the C4′ position of the internal lysine-PLP aldimine of GABA-AT with its amino group (Scheme 3). After the formation of the external aldimine, a dicarboxylate is generated via γ-deprotonation of the cyclopentane ring, followed by hydrolysis of the difluorovinyl group¹⁰. Once generated, the dicarboxylate adduct can interact with Arg445 to form a tight-binding conformation. If not, this adduct is converted to a ketone product via its turnover mechanism¹⁰ (Scheme 3).

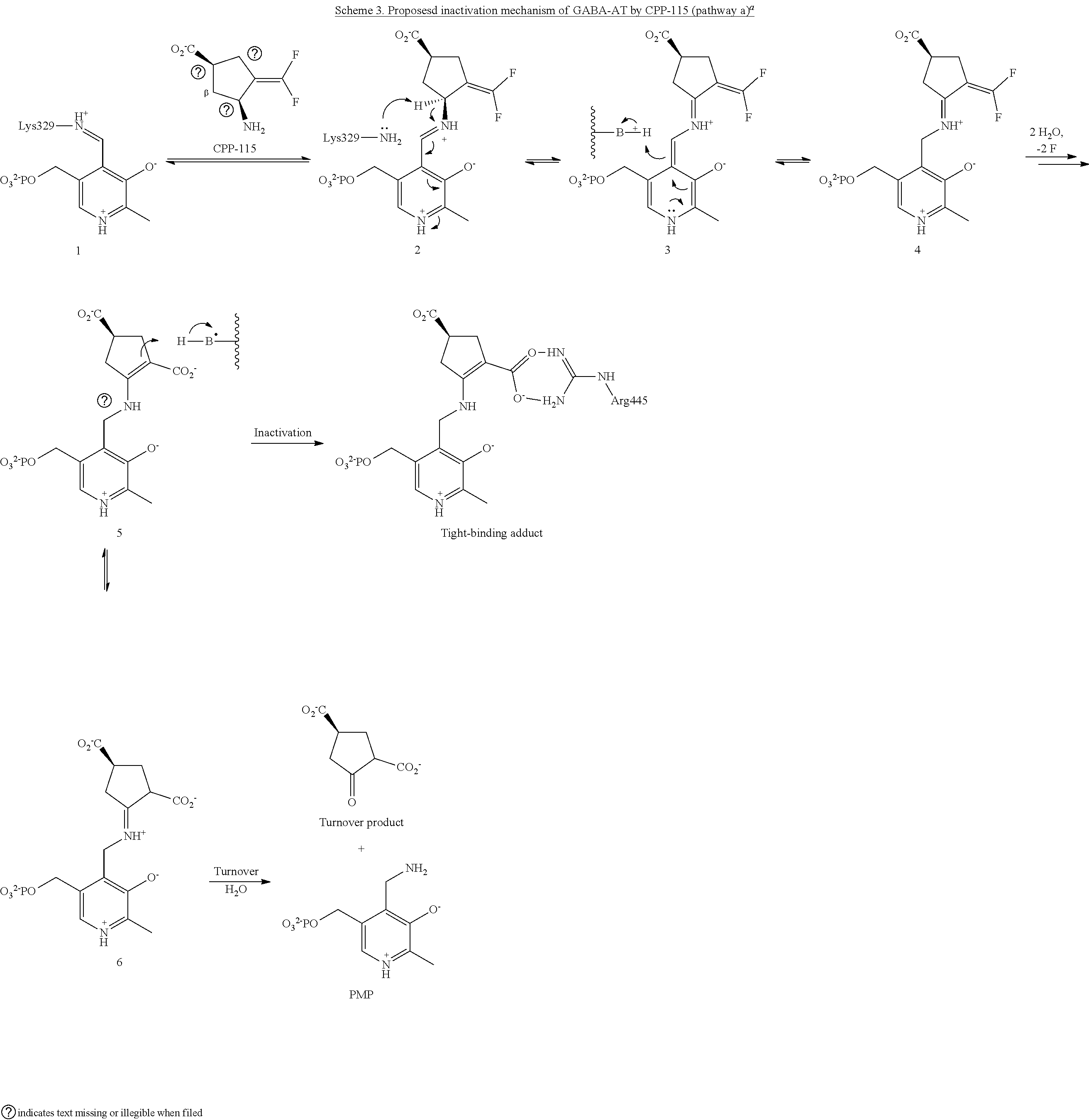

aPMP, Pyridoxamine-5-Phosphate.

Although CPP-115 proved its safety in the Phase I clinical trial, concerns about the long-term toxicity of CPP-115 and the closely related derivative OV329 have arisen owing to their off-target activity on the ornithine aminotransferase (OAT). OAT, which is classified into the same enzyme subgroup (aminotransferase subgroup Ill) as GABA-AT¹¹, generates glutamic-γ-semialdehyde by transfer of the δ-amino group of _{L}-ornithine¹² (FIG. 1B). Based on their substrate, mechanism, and active site similarity, we have reported the OAT inactivation of a few GABA-AT inactivators¹³. Unfortunately, CPP-115 exhibited OAT inactivation at high concentrations (k_{inact}/K_{I}=0.83 min⁻¹mM⁻¹). In other experiments, OV329 was also confirmed to inactivate OAT¹⁴ (k_{inact}/K_{I}=7.6 min⁻¹mM⁻¹), failing to achieve improved selectivity, despite its excellent efficiency on GABA-AT. Further, there are serious side effects with the known GABA aminotransferase inactivators, which also inhibit the enzyme ornithine aminotransferase. There remains a need for inactivators with better selectivity for GABA-AT over OAT.

### SUMMARY OF THE INVENTION

Disclosed herein are difluoromethylenyl-containing aminocyclopentanecarboxylic acid analogues and methods of using the same. In some embodiments, the disclosed compounds may inactivate GABA aminotransferase and may be used methods of treating neurological disease or disorder, pain, and/or cancer.

One aspect of the present disclosure provides a compound of formula (I), or a salt or hydrate thereof,

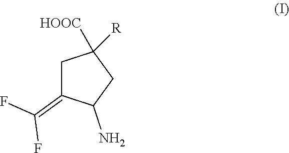

wherein
R is C₁-C₆-alkyl, C₂-C₆-alkenyl, or C₂-C₆-alkynyl, wherein the C₁-C₆-alkyl is optionally substituted with halogen or hydroxyl.

Another aspect of the present disclosure provides a pharmaceutical composition comprising the compound disclosed herein and a pharmaceutically acceptable carrier, diluent, or excipient.

Another aspect of the present disclosure provides a method of modulating γ-aminobutyric acid aminotransferase (GABA-AT) activity, the method comprising contacting the compound disclosed herein, or a salt or hydrate thereof, or the pharmaceutical composition disclosed herein, with a medium comprising a GABA-AT, wherein the compound or the composition is present in an amount sufficient to modulate GABA-AT activity.

Another aspect of the present disclosure provides a method for treating a disease or disorder associated with GABA-AT activity in a subject in need thereof, the method comprising administering to the subject a therapeutically effective amount of the compound disclosed herein, or a salt or hydrate thereof, or the pharmaceutical composition disclosed herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows enzymatic catalysis by (A) GABA-AT and (B) OAT. GABA, 7-aminobutyric acid; GABA-AT, GABA aminotransferase; PLP, pyridoxal-5-phosphate; PMP, pyridoxamine-5-phosphate; OAT, ornithine aminotransferase.

FIG. 2 shows the structures of vigabatrin, CPP-115, and OV329.

FIG. 3 shows the conformations of cyclopentene ring in the model of PLP-imine intermediate (A) 7 (R═H) and (B) 7′ (R=Me).

FIG. 4 shows the N_{Lys292}—C4′ distances from the MD simulation trajectories of 7 and the corresponding α-methyl derivative in the hOAT model.

FIG. 5 shows the N_{Lys292}—O_{*Thr322} and O_{*Thr322}—H_{β} distances from the MD simulation trajectories of 7 and the corresponding α-methyl derivative in the hOAT model.

FIG. 6 shows the calculated binding free energy of the external aldimine intermediate for each inactivator in GABA-AT and OAT. The results are presented as mean±SEM (n=6). The asterisk and N.S. denote p<0.05 and not significant by two-sample t-test, respectively.

FIG. 7 shows the side chain composition around cyclopentane ring of CPP-115 external aldimine complex in (A) GABA-AT and (B) OAT.

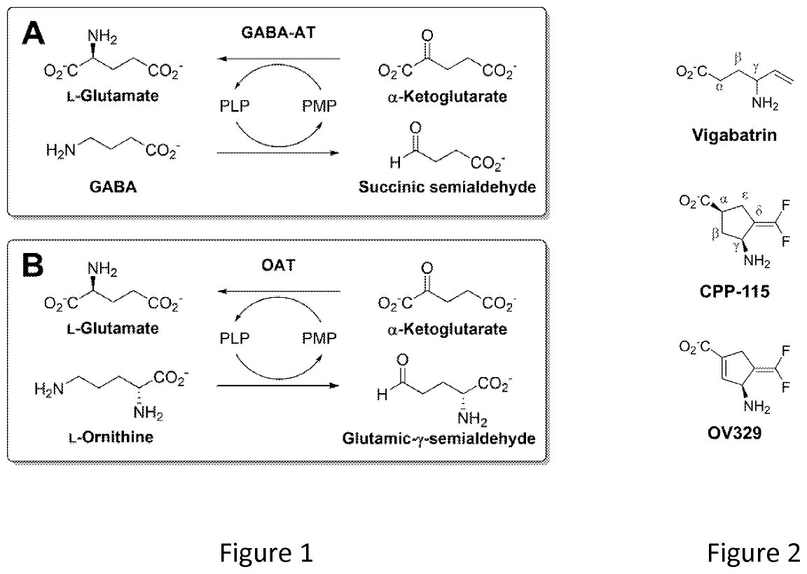

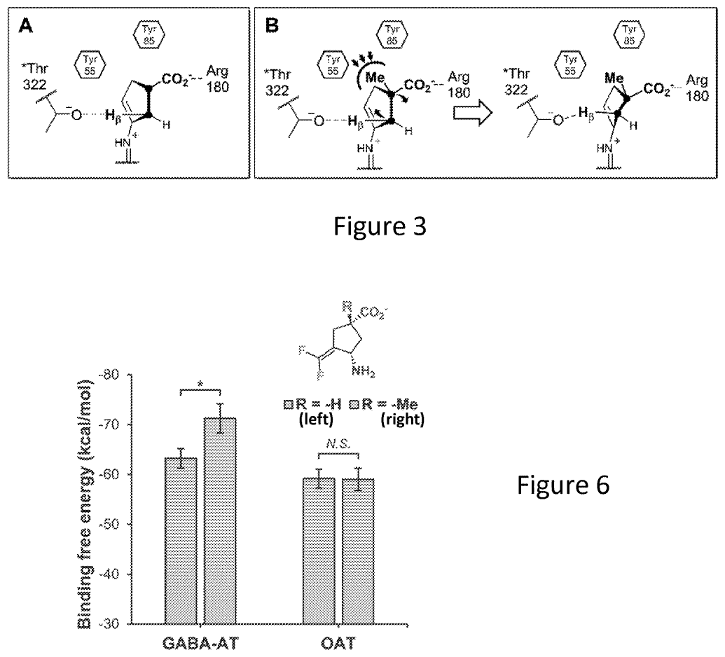

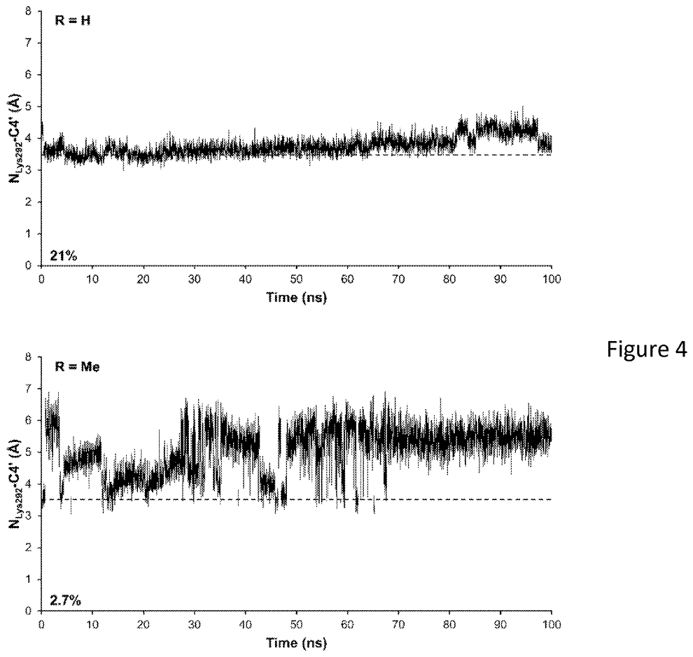

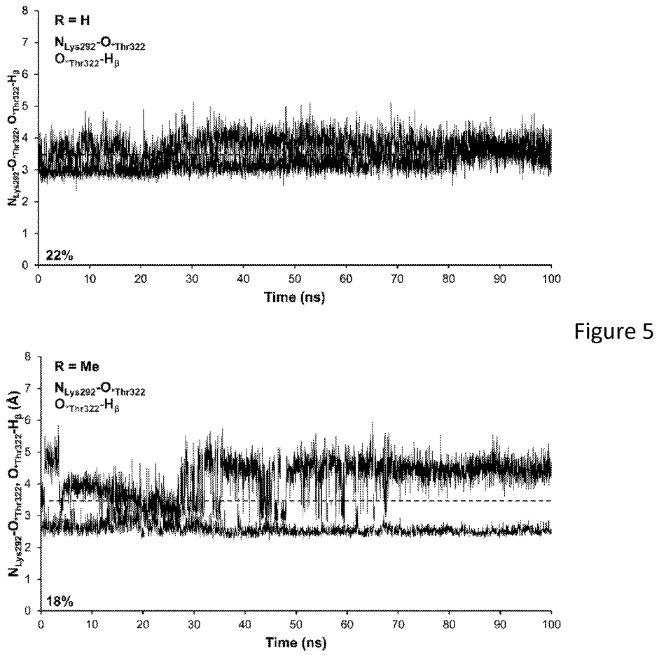

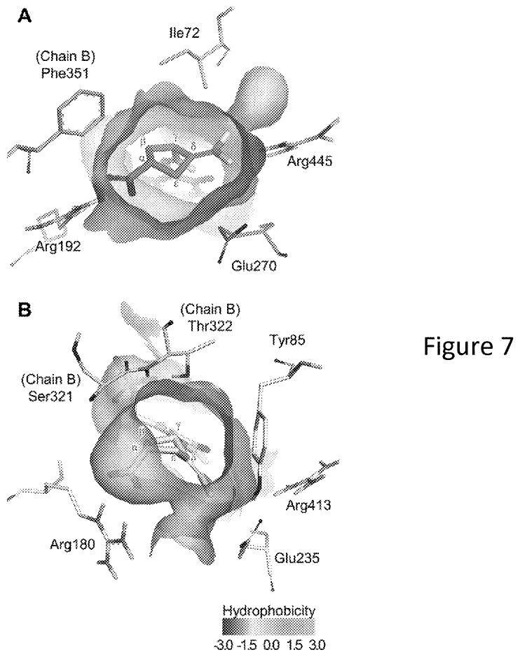

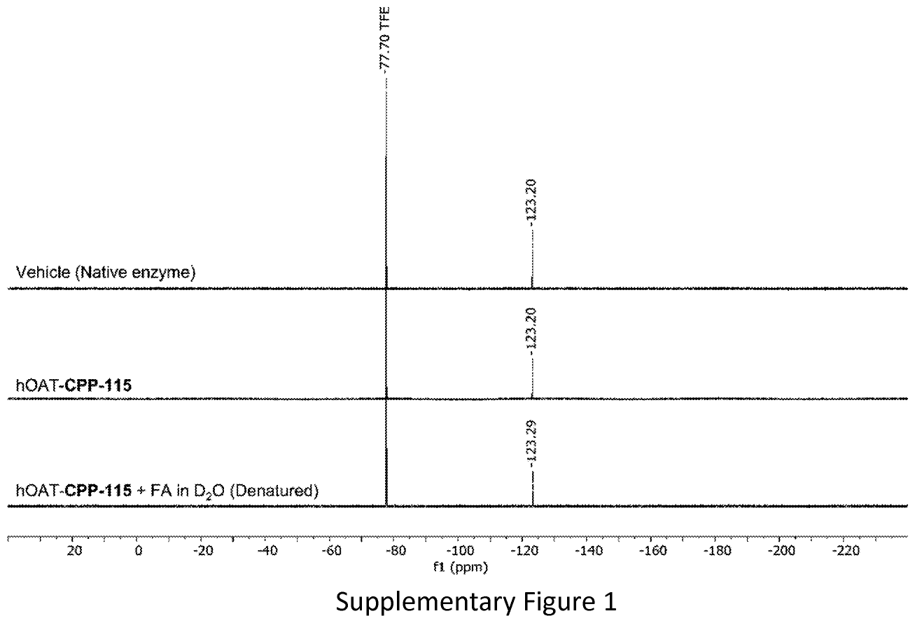

Supplementary FIG. 1 shows the ¹⁹F-NMR data of native hOAT and inactivated hOAT by CPP-115.

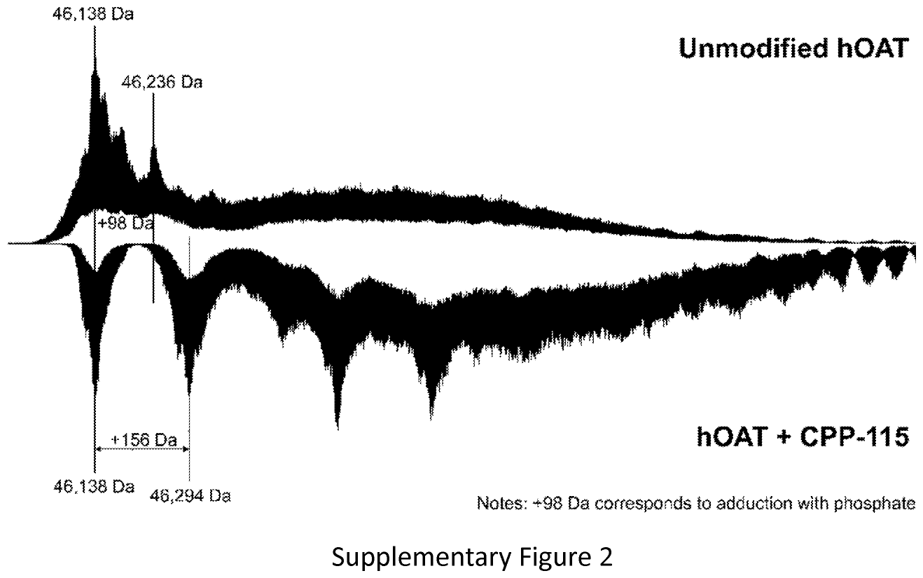

Supplementary FIG. 2 shows the native protein MS results from hOAT and CPP-115.

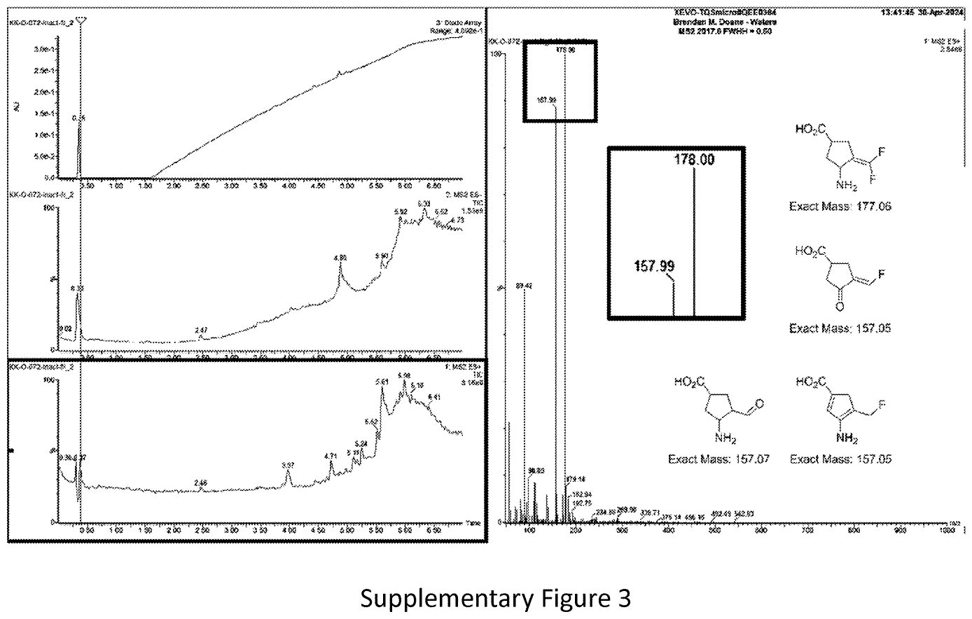

Supplementary FIG. 3 shows MS result from the metabolite of CPP-115 in hOAT.

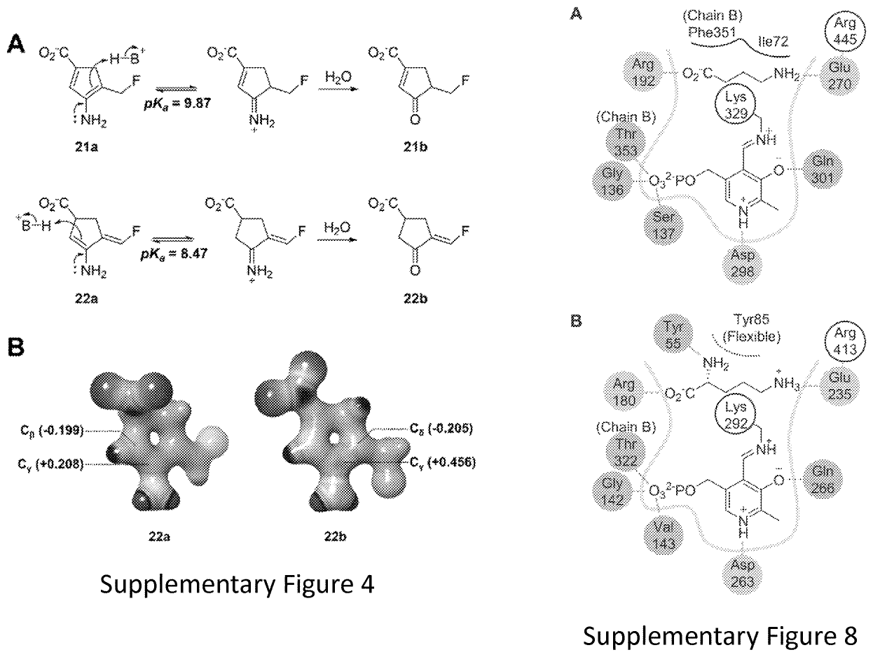

Supplementary FIG. 4 shows theoretical pKₐ calculations of the conjugate acid of 21a.

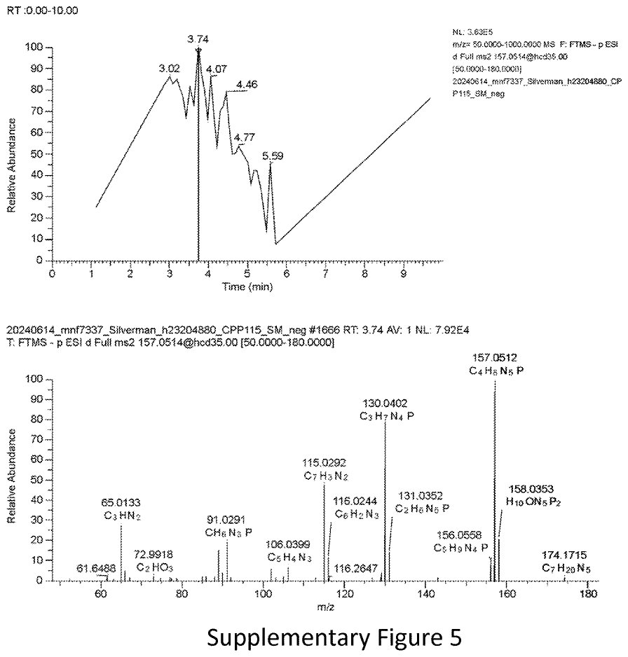

Supplementary FIG. 5 shows the high-resolution MS result from the metabolite of CPP-115 in hOAT.

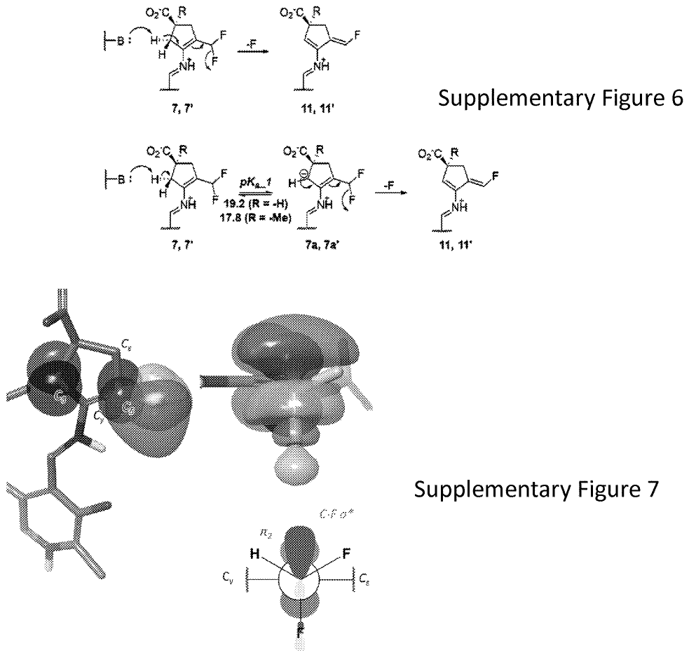

Supplementary FIG. 6 shows theoretical pKa calculation for PLP-imine intermediate 7 (R═H) and 7′ (R=Me).

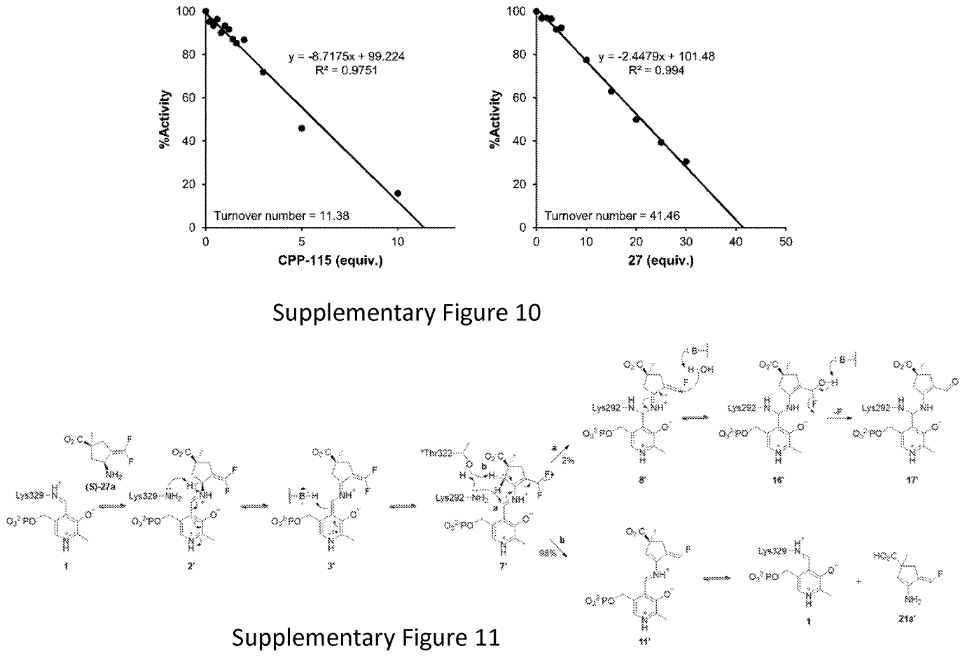

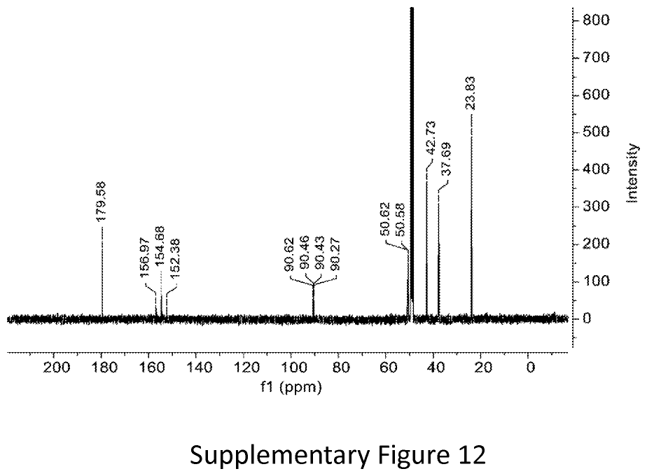

Supplementary FIG. 7 shows the molecular orbitals for fluorine release-facilitated β-deprotonation.

Supplementary FIG. 8 shows the two-dimensional representations for binding mode of PLP in (A) GABA-AT³⁰ (PDB ID: 1OHV) and (B) OAT³¹ (PDB ID: 1OAT). Hydrogen bonds and salt bridges are shown as green and orange dotted lines, respectively. For clarity, only polar interactions were depicted.

Supplementary FIG. 10 shows the turnover number calculation of CPP-115 and (S)-27a in OAT.

Supplementary FIG. 11 shows the possible pathways of (S)-27a in OAT.

Supplementary FIG. 12 shows the ¹³C NMR spectrum (CD₃OD) of (1S,3S)-3-Amino-4-(difluoromethylene)-1-methylcyclopentane-1-carboxylic acid hydrochloride (29).

### DETAILED DESCRIPTION OF THE INVENTION

Disclosed herein are a series of new compounds designed based on the suggested mechanisms and computational studies including docking, molecular dynamic (MD) simulation, quantum mechanical calculation, and molecular mechanics with generalized Born and surface area (MM-GBSA) techniques. The newly designed compound and its derivatives were synthesized and evaluated in vitro to determine their kinetic profiles for GABA-AT and OAT inactivation. The disclosed compounds exhibited selectivity for GABA-AT over OAT. The disclosed compounds and compositions may be utilized in methods of treating a disease or disorder associated with GABA-AT activity, such as epilepsies, neuropathic pain, and inflammatory pain.

### Chemical Entities

The term “alkyl” refers to a straight-chain, branched, or cyclized alkyl radical in all its isomeric forms, such as a straight or branched group of 1-12, 1-10, or 1-6 carbon atoms, referred to herein as C₁-C₁₂-alkyl, C₁-C₁₀-alkyl, and C₁-C₆-alkyl, respectively.

The term “alkylene” refers to a diradical of straight-chain or branched alkyl group (e.g., a diradical of straight-chain or branched C₁-C₁₂ alkyl group). Exemplary alkylene groups include, but are not limited to —CH₂—, —CH₂CH₂—, —CH₂CH₂CH₂—, —CH(CH₃)CH₂—, —CH₂CH(CH₃)CH₂—, —CH(CH₂CH₃)CH₂—, and the like.

The term “alkenyl” refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The term “alkynyl” refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The terms “alkoxy” or “alkoxyl” refers to an alkyl group, as defined above, having an oxygen radical attached thereto. Representative alkoxy groups include methoxy, ethoxy, tert-butoxy and the like.

The term “cycloalkyl” refers to a monovalent saturated or partially saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, the cycloalkyl group is not substituted, i.e., it is unsubstituted.

The term “heterocycloalkyl” (or “heterocyclyl”) refers to a monovalent saturated or partially saturated cyclic, bicyclic, or bridged cyclic hydrocarbon group of 3-12, 3-8, 4-8, or 4-6 carbons in which at least one carbon of the cycloalkane is replaced with a heteroatom such as, for example, N, O, and/or S.

The terms “halo” and “halogen” refer to a halogen atom or halogen radical (e.g., —F, —Cl, —Br, or —I).

The terms “hydroxyl” refers to —OH.

The term “haloalkyl” refers to an alkyl group that is substituted with at least one halogen. For example, —CH₂F, —CHF₂, —CF₃, —CH₂CF₃, —CF₂CF₃, and the like.

The term “cyano” refers to —CN.

The term “aryl” refers to a carbocyclic aromatic group. The term “aryl” includes monocyclic ring systems, and polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aryl ring is unsubstituted. In certain embodiments, the aryl group is a 6-10 membered ring structure. Representative aryl groups include phenyl, naphthyl, anthracenyl, 1,3-benzodioxolyl and the like.

The substituents on the aryl (e.g., phenyl) rings in the compounds of the disclosure may be at ortho-, meta-, or para-positions.

The term “heteroaryl” refers to an aromatic 5- to 10-membered ring structure, alternatively 6- to 10-membered rings, whose ring structures include one to four heteroatoms, such as nitrogen, oxygen, and sulfur. The number of ring atoms in the heteroaryl group can be specified using Cx-Cx nomenclature where x is an integer specifying the number of ring atoms. For example, a C₃-C₇ heteroaryl group refers to an aromatic 3- to 7-membered ring structure containing one to four heteroatoms, such as nitrogen, oxygen, and sulfur. The term “heteroaryl” includes monocyclic ring systems, and polycyclic ring systems having two or more heterocyclic rings in which two or more carbon or heteroatom are common to two adjoining rings (the rings are “fused rings”) wherein at least one of the rings is a heterocyclic aromatic group and, e.g., the other ring(s) may be cycloalkyls, cycloalkenyls, cycloalkynyls, and/or aryls. Representative heteroaryl groups include pyridinyl, quinolinyl, furanyl, thionyl, indolyl, and the like.

The term “optionally substituted” refers to one or more carbon atoms in the group being independently substituted with one or more functional groups described herein.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. The present invention encompasses various stereo isomers of these compounds and mixtures thereof. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound, which composition may comprise, consist essentially of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

As used herein, “salt” refers to acid addition salts and basic addition salts. It may also refer to those salts that may be prepared in situ during the final isolation and purification of the compounds of the invention. In some embodiments, “salt” includes HCl salt of the compound described herein.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### Compounds

One aspect of the present disclosure provides a compound of formula (I) or a salt or hydrate thereof,

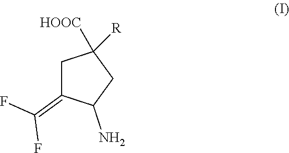

wherein
R is C₁-C₆-alkyl, C₂-C₆-alkenyl, or C₂-C₆-alkynyl, wherein the C₁-C₆-alkyl is optionally substituted with halogen or hydroxyl.

In some embodiments, the compound is a compound of formula (Ia-A) or (Ia-B), or a salt or hydrate thereof.

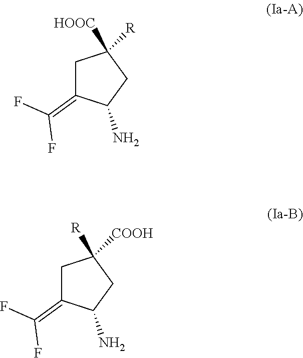

In some embodiments, R is C₁-C₆-alkyl optionally substituted with halogen or hydroxyl. In some such embodiments, R may be methyl, ethyl, n-propyl, i-butyl, —CH₂F, —CH₂Cl, or —CH₂OH.

In some embodiments, R is —CH═CH₂ or —C≡CH.

In some embodiments, the compound is

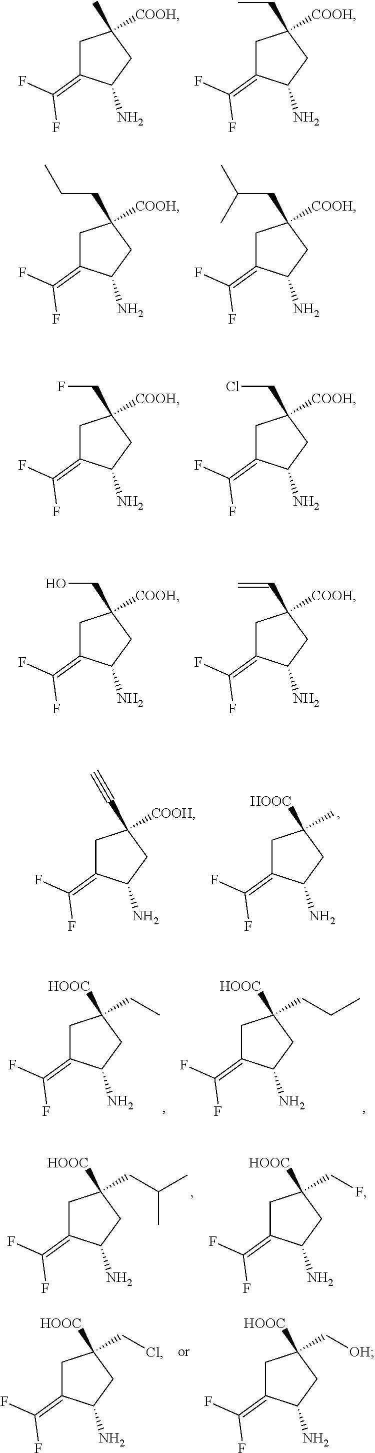

or a salt or hydrate thereof.

In some embodiments, the compound is

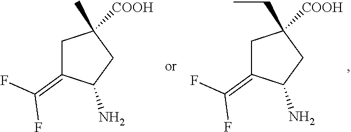

or a salt or hydrate thereof.

The disclosed compounds may exhibit one or more biological activities. In some embodiments, the disclosed compounds modulate the activity of γ-aminobutyric acid aminotransferase (GABA-AT) and/or ornithine aminotransferase (OAT). In some embodiments, the disclosed compounds inhibit the activity of GABA-AT and/or OAT. In some embodiments, the disclosed compounds inhibit the activity of GABA-AT and/or OAT by at least 50%, 60%, 70%, 80%, 90%, 95%, 96%, 97%, 98%, or 99% at a concentration of less than 100 μM, 50 μM, 10 μM, 1 μM, 0.1 μM, 0.05 μM, 0.01 μM, 0.005 μM, 0.001 μM, 0.1 nM, 0.01 nM, or less. In some embodiments, the efficiency of the inactivation of GABA-AT and/or OAT may be determined using kinetic constants for the inactivation of GABA-AT and/or OAT, such as through the values of K_{I}, k_{inact}, and k_{inact}/K_{I}.

In some embodiments, the disclosed compound is a selective GABA-AT inactivator. The disclosed compound may have a selectivity for GABA-AT versus OAT of at least 10, 15, 20, 30, 40, 50, 100, 150, 200, 250, or higher. In some embodiments, the selectivity for GABA-AT versus OAT may be assessed by comparing the k_{inact}/K_{I} ratios from inactivation of GABA-AT and OAT, as disclosed herein.

In some embodiments, the disclosed compound exhibits lower toxicity and/or cause less side effects after being administered to a subject, compared to a reference compound. The reference compound may have a lower selectivity for GABA-AT versus OAT, compared to the compound disclosed herein.

Another aspect of the present disclosure provides a salt of the compound disclosed herein. The salt may have a formula of (Ib-A) or (Ib-B).

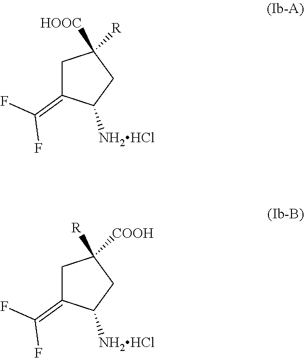

### Pharmaceutical Compositions

Another aspect of the present disclosure provides a pharmaceutical composition comprising the compound disclosed herein and a pharmaceutically acceptable carrier, diluent, or excipient.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

In some embodiments, the compounds disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 nM, 0.005 nM, 0.01 nM, 0.5 nM, 0.1 nM, 1.0 nM, 10 nM, and 100 nM (e.g., 0.1 nM-1.0 nM).

It is understood by those skilled in the art that dosage amount will vary with the activity of a particular inhibitor compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically acceptable salt thereof, or related composition may be administered in two or more sub-doses, administered separately over an appropriate period of time.

The compounds for use according to the methods of disclosed herein may be administered as a single compound or a combination of compounds. For example, a compound that modulates γ-Aminobutyric acid aminotransferase (GABA-AT) activity and/or ornithine aminotransferase (OAT) activity may be administered as a single compound or in combination with another compound that modulates GABA-AT activity and/or ornithine aminotransferase (OAT) activity or that has a different pharmacological activity. In some embodiments, one or more additional therapeutic agents may be administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition is formulated to comprise the disclosed compounds and further to comprise one or more additional therapeutic agents, for example, one or more additional therapeutic agents for treating diseases and disorders.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases. In some embodiments, HCl salts of the disclosed compounds are provided.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range of from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 nM, 0.005 nM, 0.01 nM, 0.5 nM, 0.1 nM, 1.0 nM, 10 nM, and 100 nM (e.g., 0.1 nM-1.0 nM).

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and crosslinked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Methods of preparing pharmaceutical formulations or compositions include the step of bringing a disclosed compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, PA.

Regardless of composition or formulation, those skilled in the art will recognize various avenues for medicament administration, together with corresponding factors and parameters to be considered in rendering such a medicament suitable for administration.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

### Methods

The disclosed compounds and pharmaceutical compositions comprising the disclosed compounds may be administered in methods of treating a disease or a disorder in a subject in need thereof, such as in methods of treating a disease or disorder associated with GABA-AT activity.

Another aspect of the present disclosure provides a method of treating a disease or disorder associated with GABA-AT activity in a subject in need thereof, the method comprising administering to the subject a therapeutically effective amount of the compound disclosed herein, or the pharmaceutical composition disclosed herein.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

A “subject in need thereof” as utilized herein refers to a subject in need of prevention and/or treatment for a disease or disorder associated with GABA-AT activity. The term “subject” may be used interchangeably with the terms “individual” and “patient” and includes human and non-human mammalian subjects. In some embodiments, the treated subject may be a mammalian subject. Although the methods disclosed herein are particularly intended for the treatment of humans, other mammals are included. By way of non-limiting examples, mammalian subjects include monkeys, equines, cattle, canines, felines, mice, rats and pigs.

As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

In some embodiments, the disease or disorder, such as the disease or disorder treated by GABA-AT inhibition, is a neurological disease or disorder, pain, or cancer. In some embodiments, the disease or disorder include, but are not limited to, epilepsy, neuropathic pain, inflammatory pain, Alzheimer's disease, Parkinson's disease, neuropathic pain, partial seizures, complex partial seizures, secondary generalized seizures, tonic-clonic seizures, succinic semialdehyde dehydrogenase deficiency (SSADHD), infantile spasms in West's syndrome, Lennox-Gastaut syndrome, tubulous sclerosis, Tourette's syndrome, movement disorders, fibromyalgia, migraine related to epilepsy, restless leg syndrome and post-traumatic stress disorder, addiction, obesity, obsessive-compulsive disorders.

In some embodiments, the disease or disorder is selected from the group consisting of epilepsy, neuropathic pain, inflammatory pain, Tourette syndrome, and disorders that cause seizures, such as traumatic brain injury, brain tumors, neurodegenerative diseases, cerebral palsy, autism, Dravet syndrome, tuberous sclerosis complex, Lafora disease.

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg 20 mg 22.5 mg, 25 mg 27.5 mg, 30 mg, 32.5 mg, 35 mg, 37.5 mg 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as end-points any of these disclosed doses (e.g., 2.5 mg-200 mg).

In some embodiments of the disclosed treatment methods, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, 20000, 30000, 40000, or 50000 ng/kg body weight of the subject. Minimal and/or maximal dose levels of the compounds for achieving therapy in the disclosed methods of treatment may include dose levels falling within ranges having as end-points any of these disclosed dose levels (e.g., 500-2000 ng/kg body weight of the subject).

As used herein the term “effective amount” refers to the amount or dose of the compound or composition, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with GABA-AT activity.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

Another aspect of the present disclosure provides a method of modulating γ-aminobutyric acid aminotransferase (GABA-AT) activity, the method comprising contacting the compound disclosed herein, or the pharmaceutical composition disclosed herein, with a medium comprising a GABA-AT, wherein the compound or the composition is present in an amount sufficient to modulate GABA-AT activity.

In some embodiments, the contact is in vivo.

“Contacting” means that a GABA-AT and one or more inhibitor compounds are brought together for purpose of binding, complexing, and/or reacting such an inhibitor compound to the enzyme. Amounts of a compound effective to inhibit a GABA-AT may be determined empirically, and making such determinations is within the skill in the art. Inhibition or otherwise affecting a GABA-AT activity includes reduction, mitigation and/or modulation, as well as elimination of GABA-AT activity, glutamate production, glutamine synthesis, cell proliferation and/or tumor growth.

The compounds and compositions disclosed herein may be administered in methods of treatment as known in the art. Accordingly, various such compounds and compositions can be administered in conjunction with such a method in any suitable way. For example, administration may comprise oral, intravenous, intraarterial, intramuscular, subcutaneous, intraperitoneal, parenteral, transdermal, intravaginal, intranasal, mucosal, sublingual, topical, rectal or subcutaneous administration, or any combination thereof.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and are not intended to limit the scope of the claimed subject matter.

### Results and Discussion

Design of a New Inactivator with GABA-AT Selectivity Over OAT

To enhance the GABA-AT selectivity over OAT, facilitating its turnover in OAT could be one of the effective strategies. We hypothesized that the fate of the inactivator proceeding to inactivation or turnover was suggested to be determined in PLP-imine intermediate 7, via lysine attack at the C4′-position and by β-deprotonation, respectively (Scheme 4). Thus, a new compound was designed to facilitate the β-deprotonation in the intermediate 7.

Initially, the α-methyl compound was designed to facilitate turnover by reducing the distance between the hydroxyl group of *Thr322 (O_{*Thr322}) and the Hp. As depicted in FIG. 3, the α-methyl group could repel itself from the side chains of Tyr55 and Tyr85, which define the space around the α-position. The repulsion was expected to push α-carbon (C_{α}) away from the tyrosine side chains and change the inactivator's cyclopentene ring conformation to compensate for the displacement of C_{α}. As driven by this broken ring planarity, it was supposed to induce a rotation of the C_{α}-C_{β} bond to move C_{β} toward O_{*Thr322}, placing O_{*Thr322} and H_{β} closer (FIG. 3B).

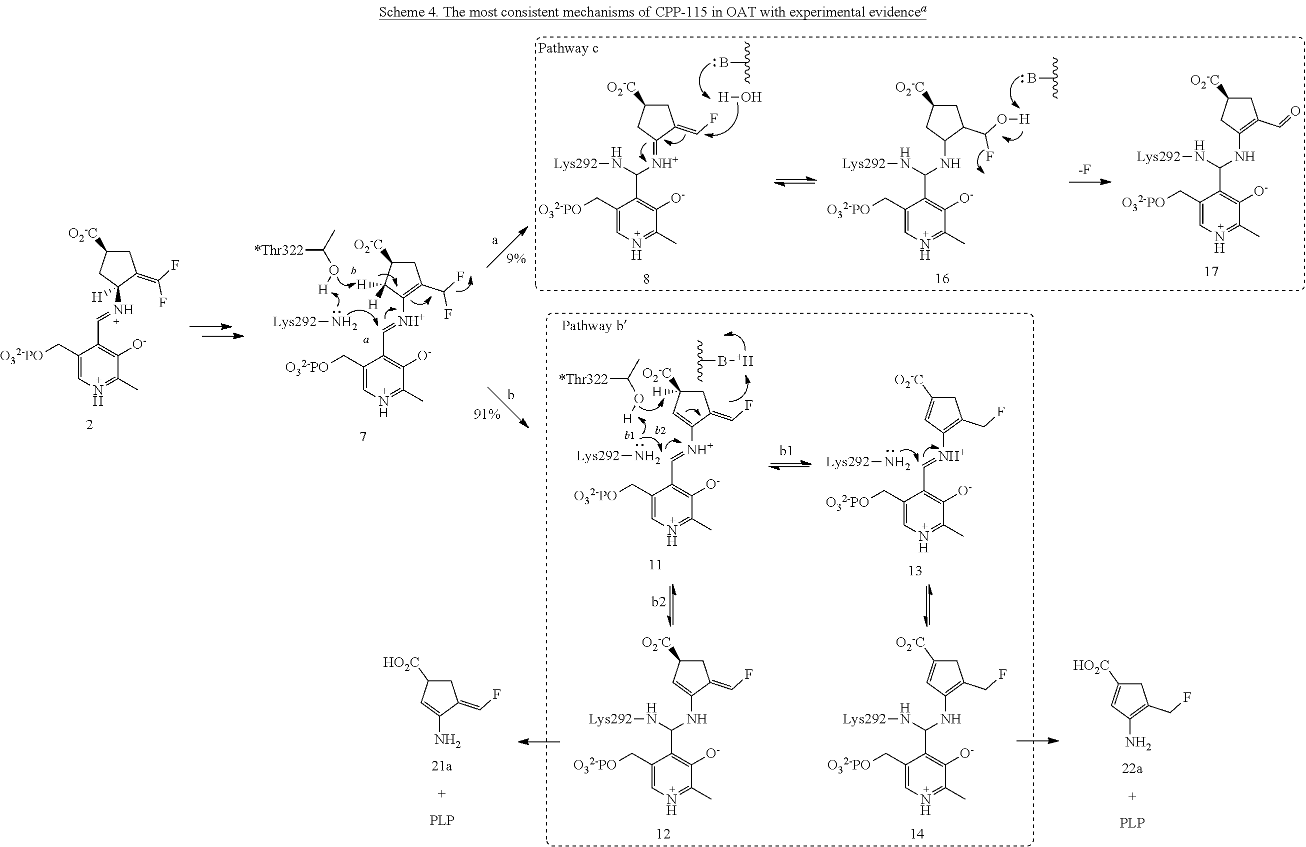

^{a}PLP, Pyridoxal-5-Phosphate

To collectively evaluate and compare possibilities for each mechanism in the intermediates of CPP-115 and the α-methyl-substituted compound in geometric aspect, the following factors were monitored in trajectories from the NMD simulation result of each corresponding intermediate model in hOAT. For the mechanism of inactivation, the distance between N_{Lys292} (the amino group of Lys292) and C4′ was measured considering the near attack conformation (NAC) hypothesis. Additionally, a concept of the Bürgi-Dunitz angle (BD angle) was introduced to create geometric criteria for the lysine attack to C4′ imine (the N_{Lys292}—C4′=N angle), even though it was initially created to explain the nucleophilic attack of a carbonyl group (the nucleophile-C═O angle).

The distance cutoff for a successful N_{Lys292}—C4′ attack was set as <3.5 Å, adopting the relaxed NAC criteria¹⁹ considering a potential discrepancy between the physiological environment and computational force field. As well as the distance criterion (N_{Lys292}—C4′<3.5 Å), when the angle is within the relaxed BD angle range (105±150), the frame was counted as an appropriate population for N_{Lys292}—C4′ attack and corresponding inactivation pathway (% inact in Table 1).

TABLE 1
The populations satisfying each geometry criterion and appropriate
population for each pathway from the MD simulation trajectories
of 7 and the corresponding α-methyl derivative in the hOAT model

| Criteria or population | CPP-115 | α-methyl compound |
|---|---|---|
| N_{Lys292}-C4′ < 3.5 Å | 21% | 2.7% |
| BD angle = 105 ± 15° | 41% | 28% |
| % inact | 4.1% | 1.7% |
| N_{Lys292}-O*_{Thr322} and | 22% | 18% |
| O*_{Thr322}-H_{β} < 3.5 Å | | |
| CF₂H dihedral = 90 ± 15° | 20% | 45% |
| % TO | 4.2% | 6.6% |

To set the criteria for β-deprotonation and related turnover pathway, the theoretical pKₐ value of the H_{β} in the intermediate 7 was calculated using a quantum mechanical method with the geometry-optimized structure using B3LYP/6-31G* level of theory followed by a single-point energy calculation with cc-pVTZ(+) basis set and the solvation free energy calculation. The theoretical pKₐ of carbanion 7a, a β-deprotonated conjugate acid of 7, was 19.2 (Supplementary FIG. 6); thus, despite the activation of *Thr322 by lysine, the pKa was too high and the β-deprotonation seems improbable. However, the β-deprotonation could be facilitated by the concerted E2-like elimination of a fluoride ion in the difluoromethyl group at the δ-position, when the highest occupied molecular orbital (HOMO) of allyl anion at C_{β}, C_{γ}, and C_{δ} (π₂) and the lowest unoccupied molecular orbital (LUMO) of C—F bond (σ*) are well-aligned (Supplementary FIG. 7).

Considering the calculated shape of the σ* and π₂ orbitals and their maximal overlap, the dihedral angle between the C—F bond and C_{γ}-C_{δ} should be perpendicular (Supplementary FIG. 7) allowing the adequate alignment of the HOMO of allyl anion and the LUMO of the C—F bond. Therefore, the range of the dihedral angle (C_{γ}-C_{δ}-C—F) for the fluorine release-facilitated β-deprotonation was set as 90±15°. Since the *Thr322 hydroxyl group should be activated by the catalytic lysine, the population for the β-deprotonation and turnover pathway (% TO in Table 1) was then counted by the NAC criteria, considering the distances N_{Lys292}—O_{*Thr322} and O_{*Thr322}—H_{β} and the angle criteria, similar to % inact.

As a result, the ratio of % TO over % inact, which sketches the partition ratio in geometric aspect, were 1.0 and 3.9 for CPP-115 and the α-methyl compound (Table 1), respectively. This result could suggest the potential 4-fold enhancement in turnover rate with the α-methyl compound. Notably, this change was mainly driven by the drastic increase in N_{Lys292}—C4′ distance (FIG. 4), while O_{*Thr322}—H_{β} distances were similar in both models (FIG. 5). In the initial design stage, the α-methyl group was supposed to move C_{β} toward O_{*Thr322} under the assumption that the PLP part of the intermediate is tightly fixed by its binding residue. However, there was slight space around the PLP part, which might allow the bending motion throughout the intermediate, not limited to the cyclopentene ring. Therefore, by this presumable motion of C4′ against N_{Lys292}, the population meeting the distance criterion could be decreased from 21% to 2.7% for the α-methyl compound compared to CPP-115.

The change in binding affinities of the α-methyl compound to GABA-AT and OAT was then predicted by post-docking rescoring of the corresponding external aldimine intermediate of each inactivator. To compare the binding affinities of GABA-AT and OAT inactivators, the corresponding external aldimine intermediate complexed in each aminotransferase was used since the inactivator quickly forms the external aldimine immediately after binding¹⁰, and the γ-deprotonation of the external aldimine is considered as a rate-limiting step²⁰. Each binding mode of the external aldimine in GABA-AT and OAT was generated by docking in the final product complex structures from X-ray crystallography of CPP-115 in GABA-AT (PDB ID: 4YOD)¹⁰ and the OAT structure that was previously used for the model generation (PDB ID: 7TED)¹⁵. Starting from the minimized complexes of each predicted binding mode, 20 ns MD simulations were conducted to generate and analyze trajectories. From the generated trajectories, several frames with stable binding modes were extracted, and the theoretical binding free energy was calculated for each frame using the MM-GBSA method.

As a result, the α-methyl compound was evaluated to significantly improve the binding free energy for GABA-AT (FIG. 6). However, this substitution failed to exhibit a significant difference in binding free energy in OAT, suggesting that methyl substitution might improve the binding affinity only for GABA-AT, approximately 10 kcal/mol in the mean difference. This result was interpreted as originating from the difference in the side chain composition around the α-position of each aminotransferase. In OAT, a pocket located at this position is defined by Tyr55 and Tyr85. However, in GABA-AT, there was a pocket bordered by *Phe174 (from the other chain) and Ile74 at the same location, and it was relatively hydrophobic compared to that in OAT (FIG. 7A); therefore, the methyl group at the α-position is able to have enhanced interaction with GABA-AT.

### Synthesis and Biological Evaluation of a Newly Designed α-Methyl Derivative

Since the proposed α-methyl compound was predicted to improve its selectivity for GABA-AT by reducing its efficiency in OAT as well as increasing its binding affinity in GABA-AT, the α-methyl derivative (S)-27a was synthesized as depicted in Scheme 5. The synthesis of (S)-27a was started from CPP-115 hydrochloride salt (24) following through the steps for its α-phenylselenylation, as previously reported⁶. The carboxylate and amino groups on CPP-115 were protected by ethyl ester and tert-butyloxycarbonyl groups, respectively. The enolate of protected intermediate 25 was then generated and followed by electrophilic methylation, resulting in (S)-26a and its diastereomer ((R)-27a) with approximately a 2.5:1 ratio. The absolute stereochemistry of each diastereomer was determined by NOESY. As a result, the major one was confirmed as the desired diastereomer, as it showed proximity between its γ-proton and the newly introduced methyl group, but its other diastereomer did not. Then, (S)-26a was further deprotected to afford (S)-27a as its hydrochloride salt form.

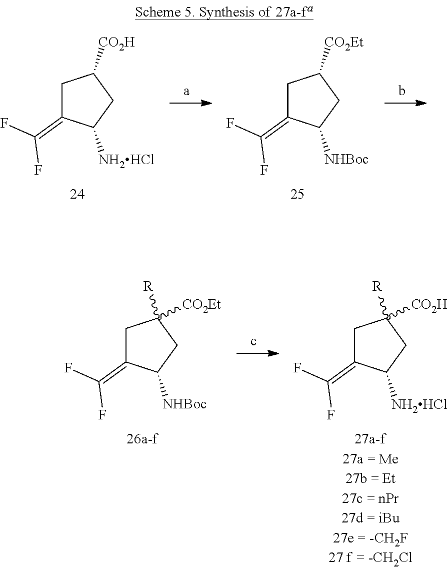

^{a}Reagents and conditions. (a) (i) acetyl chloride, ethanol, 0 °C to rt, 1 h; (ii) triethylamine, di-tert-butyl dicarbonate, rt, 1 h, 86%; (b) (i) KHMDS, THF, −78 °C, 2 h; (ii) various alkyl iodides, THF, −78 °C, 3 h; (c) 4 N HCl (aq.), 1,4-dioxane, 60 °C, 20 h, 14-43% for 2 steps.

From the in vitro enzyme assay results (Table 2), (S)-27a showed a similar K_{I} value (1.7±0.7 μM) to that of CPP-115 (and 1.1±0.5 μM) for GABA-AT; however, the k_{inact} of (S)-27a was higher than that of CPP-115 (0.10±0.02 min⁻¹ and 0.015±0.002 min⁻¹, respectively), thus the efficiency of the inactivator (k_{inact}/K_{I} ratio) was increased by approximately 4 times (57 vs 14 min⁻¹mM⁻¹).

TABLE 2
Kinetic constants for the inactivation of GABA-AT and OAT

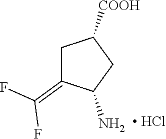 [SMILES: N[C@H]1C[C@@H](C(=O)O)CC1=C(F)F ; "· HCl"]

CPP-115 (24)

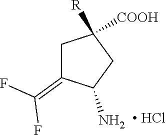

(S)-27a-f, 39, 33, 35

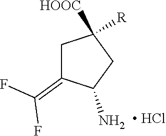

(R)-27a-f, 29

| | | GABA-AT | | | OAT | | | |
|---|---|---|---|---|---|---|---|---|
| Com- | | k_{inact} | K_{I} | k_{inact}/K_{I} | k_{inact} | K_{I} | k_{inact}/K_{I} | |
| pound | R | (min⁻¹) | (mM) | (min⁻¹mM⁻¹) | (min⁻¹) | (mM) | (min⁻¹mM⁻¹) | Selectivity^{a} |
| CPP-115 | H | 0.20 ± 0.03 | 0.0073 ± 0.0023 | 27 | 0.017 ± 0.002 | 0.014 ± 0.005 | 1.2 | 23 |
| (24) | | | | | | | | |
| (S)-27a | Me | 0.25 ± 0.03 | 0.0051 ± 0.0010 | 50 | 0.086 ± 0.003 | 0.36 ± 0.04 | 0.24 | 210 |
| (R)-27a | | 0.092 ± 0.015 | 0.061 ± 0.026 | 1.5 | N.D.^{b} | N.D.^{b} | 17% | >1 |
| | | | | | | | at 1 mM | |
| (S)-27b | Et | 0.23 ± 0.01 | 0.048 ± 0.006 | 4.7 | 0.053 ± 0.001 | 2.3 ± 0.2 | 0.0023 | 210 |
| (R)-27b | | N.D.^{b} | N.D.^{b} | 13% at 1 mM | N.D.^{b} | N.D.^{b} | 5% | N.D.^{b} |
| | | | | | | | at 1 mM | |
| (S)-27c | nPr | ^{d} | ^{d} | ^{d} | N.D.^{b} | N.D.^{b} | N.A.^{c} | N.D.^{b} |
| | | | | | | | at 1 mM | |
| (R)-27c | | N.D.^{b} | N.D.^{b} | N.A.^{c} at 1 mM | N.D.^{b} | N.D.^{b} | N.A.^{c} | N.D.^{b} |
| | | | | | | | at 1 mM | |
| (S)-27d | iBu | N.D.^{b} | N.D.^{b} | N.A.^{c} at 1 mM | ^{d} | ^{d} | ^{d} | N.D.^{b} |
| (R)-27d | | N.D.^{b} | N.D.^{b} | N.A.^{c} at 1 mM | ^{d} | ^{d} | ^{d} | N.D.^{b} |
| (S)-27e | —CH₂F | 0.086 ± 0.003 | 0.0064 ± 0.0006 | 14 | 0.057 ± 0.005 | 0.094 ± 0.21 | 0.61 | 23 |
| (R)-27e | | N.D.^{b} | N.D.^{b} | 13% at 1 mM | 0.040 ± 0.003 | 0.488 ± 0.113 | 0.081 | <1 |
| (S)-27f | —CH₂Cl | To be synthesized | | | | | | |
| (R)-27f | | ^{d} | ^{d} | ^{d} | 0.045 ± 0.006 | 0.15 ± 0.05 | 0.30 | ^{d} |
| (S)-29 | —CH₂OH | ^{d} | ^{d} | ^{d} | 0.019 ± 0.002 | 0.80 ± 0.26 | 0.024 | ^{d} |
| (R)-29 | | ^{d} | ^{d} | ^{d} | 0.016 ± 0.002 | 0.87 ± 0.31 | 0.019 | ^{d} |
| (S)-33 | —CH═CH₂ | ^{d} | ^{d} | ^{d} | 0.039 ± 0.003 | 0.093 ± 0.024 | 0.42 | ^{d} |
| (S)-35 | —C≡CH | ^{d} | ^{d} | ^{d} | 0.16 ± 0.03 | 0.030 ± 0.013 | 5.2 | ^{d} |

^{a}(k_{inact}/K_{I} for GABA-AT)/(k_{inact}/K_{I} for OAT).
^{b}Not determined without time-dependent inactivation.
^{c}No activity.
^{d}To be tested.

Additionally, for OAT, (S)-27a showed a remarkable decrease in K_{I} value (0.39±0.05 mM) compared to CPP-115 (0.015±0.005 mM), resulting in a 5-fold less efficient inactivation profile (k_{inact}=0.087±0.004; k_{inact}/K_{I}=0.22 min⁻¹ mM⁻¹) compared to CPP-115 (k_{inact}=0.017±0.002; k_{inact}/K_{I}=1.2 min⁻¹mM⁻¹). The 3.6-fold increased turnover by the α-methyl compound (11.4 and 41.5 for CPP-115 and (S)-27a, respectively) would mainly affect this reduced efficiency (Supplementary FIG. 10). Consistent with its parent molecule CPP-115, the mass spectrum for the corresponding amine turnover product 21a′ was detected from its MS result, indicating the same turnover pathway for this molecule in OAT (Supplementary FIG. 11). Overall, (S)-27a exhibited 250 times more efficient inactivation on GABA-AT over OAT by its k_{inact}/K_{I} ratios (57 vs 0.223 min⁻¹mM⁻¹), and the methyl substitution on the α-position of CPP-115 improved its selectivity more than 20 times (250 vs 12).

### Synthesis and Biological Evaluation of New α-Substituted Derivatives

Since the α-methyl compound proves to enhance GABA-AT selectivity over OAT, various moieties were introduced at the same position. For the primary alkyl substituents, the protected intermediate 25 was directly reacted with KHMDS and appropriate alkyl iodides (Scheme 5). Then, following deprotection of ethyl ester and Boc group yields (S)-27b-f. During the synthesis, the opposite diastereomers for the α-position were also obtained ((R)-27a-f).

The synthesis of hydroxymethyl derivative 29 is shown in Scheme 6. Starting from the protected intermediate 25, pivaloyloxymethyl was substituted at the α-position and all the protecting groups were then deprotected by aqueous HCl solution to yield 29. Then, the carboxylic acid and amino group in 29 was re-protected with ethyl ester and Boc-protecting group to proceed into the formyl intermediate 31, by Dess-Martin oxidation. From this formyl intermediate, vinyl (32) and ethynyl (34) intermediates were synthesized respectively by the Wittig reaction and Bestman-Ohira reaction and further deprotected to yield 33 and 35.

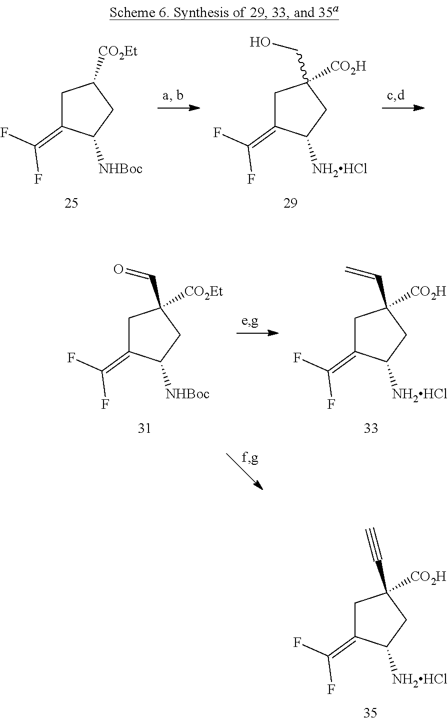

^{a}Reagents and conditions. (a)(i) KHMDS, THF, −78 °C, 2 h; (ii) iodomethyl pivalate, THF, −78 °C, 3 h; (b) 4 N HCl (aq.), 1,4-dioxane, 60 °C, 20 h, 12-31% for 2 steps; (c)(i) H₂SO₄, ethanol, 60 °C, 2 h; (ii) triethylamine, di-tert-butyl dicarbonate, rt, 1 h, 90%; (d) (i) Dess-Martin periodinane, NaHCO₃, DCM, 0 °C to rt, 2 h; (ii) Na₂S₂O₃ (aq.), NaHCO₃ (aq.), rt, 45 min, 96%; (e) KO^{t}Bu, MePPh₃Br, THF, 0 °C, 1 h, 60%; (f) K₂CO₃, dimethyl(1-diazo-2-oxopropyl)phosphonate, methanol, 0 °C, 1 h, 70%; (g) 4 N HCl (aq.), 1,4-dioxane, 60 °C, 20 h, 82-88%.

Synthesized compounds were subjected to in vitro enzyme assay in both GABA-AT and OAT and the results are shown in Table 2. Among the evaluated compounds, ethyl-substituted derivative (S)-27b showed a similar selectivity with methyl derivative (S)-27a, even with a 3-fold decreased efficiency for GABA-AT, but compensated by a 50-fold decreased efficiency for OAT. Relying on its putative higher hydrophobicity, ethyl derivative could show better pharmacokinetic profiles than (S)-27a in terms of membrane permeability. While the fluoromethyl-substituted derivative (S)-27e showed the best efficiency for GABA-AT inactivation except for (S)-27a, this molecule failed to enhance the selectivity from CPP-115 owing to its moderate efficiency in OAT. Interestingly, the opposite diastereomer of (R)-27e showed time-dependent inactivation in OAT while not in GABA-AT. It indicates that this molecule is an OAT-selective inactivator, rather than GABA-AT-selective, unlike any other α-substituted CPP-115 derivatives.

### CONCLUSION

We have reported CPP-115⁹ and OV329⁶ as efficient inactivators of GABA-AT, which is associated with various neurological conditions^{2, 3}. Unfortunately, these inactivators show undesirable inactivating activity toward OAT^{13, 14}, which has a similar substrate and active site to GABA-AT¹³. An α-methyl compound was designed. The newly designed inactivator was expected to show ~4-fold increased turnover in OAT compared to CPP-115, while predicted to bind with enhanced affinity in GABA-AT, from its theoretical binding free energy. Therefore, the α-methyl derivative ((S)-27a) was synthesized and evaluated for GABA-AT and OAT inhibitory activity and found that it exhibited more than 20 times higher GABA-AT selectivity compared to CPP-115. Additionally, various substitutions were introduced at the same position. In conclusion, we believe that this study is a valuable proof-of-concept experiment in which selectivity is significantly improved by the substituent at the α-position. Therefore, (S)-27a and (S)-27b should be further developed as a selective GABA-AT inactivator for the treatment of neurological disorders.

### Materials and Methods

### General Procedures

All reagents and solvents were obtained from commercial suppliers and used without further purification except for CPP-115⁹, synthesized at IRIX Pharmaceuticals (Florence, SC, USA). ¹H and ¹³C nuclear magnetic resonance (NMR) spectra were recorded on a Bruker Avance III spectrometer (Bruker; Billerica, MA, USA) operating at frequencies of 500 MHz and 126 MHz, respectively. The spectra were acquired in CDCl₃ and CD₃OD. Chemical shifts and coupling constants are reported in parts per million (δ) and Hz, respectively. The spectral data were presented as follows: chemical shift, multiplicity (s, singlet; d, doublet; t, triplet; q, quartet; p, pentet; dd: doublet of doublet; dt: doublet of triplet; dtd: doublet of triplet of doublet; tt, triplet of triplet; qd: quartet of doublet; qq: quartet of quartet; m, multiplet; br, broad), integration, and coupling constant. Mass spectra were obtained using a Thermo TSQ Quantum system (Thermo Fisher Scientific; Waltham, MA, USA) in the positive ion mode using atmospheric pressure chemical ionization (APCI) with the Agilent Infinity 1260 HPLC system (Agilent Technologies; Santa Clara, CA, USA) with the following conditions: Phenomenex Kintex C-18 column (50×2.1 mm, 2.6 μm); mobile phase, 5-100% acetonitrile/water containing 0.05% trifluoroacetic acid at a flow rate of 0.9 mL/min for 6 min.

### Synthesis

### Ethyl (1S,3S)-3-((tert-butoxycarbonyl)amino)-4-(difluoromethylene)cyclopentane-1-carboxylate (25)

To dry ethanol (27 mL), acetyl chloride (2.5 mL, 35 mmol) was added at 0 °C, and the mixture was stirred for 10 min followed by adding CPP-115 (1, 1.5 g, 7.0 mmol). After stirring at room temperature for 1 h, triethylamine (6.8 mL, 49 mmol) and di-tert-butyl dicarbonate (1.9 mL, 8.4 mmol) were added in sequence. Then the resulting solution was stirred for an additional 1 h at room temperature. The reaction mixture was concentrated, dissolved in ethyl acetate, and washed with 2 N HCl (aq.), saturated NaHCO₃ (aq.), and brine. The organic layer was dried over anhydrous Na₂SO₄, filtered, and concentrated in vacuo to afford 25 as a white solid (1.9 g, 86%). ¹H NMR (500 MHz, CDCl₃) δ 4.98 (s, 1H), 4.78 (s, 1H), 4.16 (qq, J=7.1, 3.7 Hz, 5H), 2.86 (p, J=7.1 Hz, 3H), 2.73-2.63 (m, 2H), 2.61-2.51 (m, 2H), 2.33 (dt, J=14.9, 7.5 Hz, 2H), 1.93 (dt, J=13.0, 6.2 Hz, 3H), 1.44 (s, 18H), 1.27 (t, J=7.2 Hz, 9H). ¹³C NMR (126 MHz, CDCl₃) δ 175.21, 155.21, 152.11 (t, J=287.2 Hz), 91.43 (t, J=18.7 Hz), 79.66, 61.15, 50.21, 41.93, 38.30 (d, J=1.8 Hz), 29.16, 28.50 (3C), 14.29. MS (ESI): [M+H]⁺=206.02.

### General Synthesis for Derivatives (S)- and (R)-27a-f; and (S)- and (R)-29

### Method A

To a solution of potassium bis(trimethylsilyl)amide solution in THF (1.0 M) at −78 °C, a solution of 25 (0.31 g, 1.0 mmol) in THF (3 mL) was added, and the mixture was stirred at −78 °C for 2 h followed by adding an appropriate alkyl iodide (2.0 mmol) in THF (3 mL). After stirring at −78 °C for 3 h, the reaction mixture was poured into saturated NH₄Cl (aq.) and extracted with ethyl acetate 3 times. Then the organic layers were combined, washed with saturated NH₄Cl (aq.) and brine, dried over anhydrous Na₂SO₄, filtered, and concentrated in vacuo. The crude mixture was purified by silica gel column chromatography to separate and afford each pure α-substituted diastereomer intermediate. Each intermediate was dissolved in 4 N HCl (aq.)/1,4-dioxane (10:1) mixture and the resulting solution was stirred at 60 °C for 20 h. The reaction mixture was concentrated in vacuo and washed with acetonitrile, if necessary, to afford (S)- and (R)-27a-d.

### Method B

α-substituted intermediates were synthesized from 25 and appropriate alkyl iodide as same as Method A. However, each intermediate was obtained as a diastereomeric mixture, and the prep-HPLC was conducted to afford pure diastereomers (S)- and (R)-27e, 27f, and 29, after the deprotection as described in Method A.

### (1S,3S)-3-amino-4-(difluoromethylene)-1-methylcyclopentane-1-carboxylic Acid ((S)-27a)

(S)-27a was synthesized from 25 and iodomethane following Method A to afford a white solid (43% for 2 steps). ¹H NMR (500 MHz, CD₃OD) δ 4.48 (tt, J=5.9, 2.0 Hz, 1H), 2.92 (dtd, J=16.0, 4.3, 1.8 Hz, 1H), 2.44 (d, J=15.9 Hz, 1H), 2.29 (dd, J=14.1, 5.8 Hz, 1H), 2.24 (dd, J=14.2, 7.8 Hz, 1H), 1.32 (s, 3H). ¹³C NMR (126 MHz, CD₃OD) δ 179.58, 157.22-152.02 (m), 90.45 (dd, J=24.4, 19.6 Hz), 50.62, 50.58, 42.73, 37.69, 23.83. MS (ESI): [M+H]⁺=192.03. MS (ESI): [M+H]⁺=192.03.

### (1R,3S)-3-amino-4-(difluoromethylene)-1-methylcyclopentane-1-carboxylic Acid ((R)-27a)

(R)-27a was synthesized from 25 and iodomethane following Method A to afford a white solid (14% for 2 steps). ¹H NMR (500 MHz, CD₃OD) δ 4.40 (td, J=8.3, 2.6 Hz, 1H), 2.90 (d, J=15.5 Hz, 1H), 2.84 (ddt, J=12.9, 8.0, 2.3 Hz, 1H), 2.43 (dddd, J=15.5, 6.4, 4.4, 1.9 Hz, 1H), 1.64 (dd, J=13.3, 8.6 Hz, 1H), 1.42 (s, 3H). MS (ESI): [M+H]⁺=192.03.

### (1S,3S)-3-amino-4-(difluoromethylene)-1-ethylcyclopentane-1-carboxylic Acid ((S)-27b)

(S)-27b was synthesized from 25 and iodoethane following Method A to afford a white solid (36% for 2 steps). ¹H NMR (500 MHz, CD₃OD) δ 4.47-4.40 (m, 1H), 2.90 (dtd, J=16.2, 4.2, 1.6 Hz, 1H), 2.49 (dt, J=16.3, 2.9 Hz, 1H), 2.35-2.20 (m, 2H), 1.84 (dq, J=14.8, 7.5 Hz, 1H), 1.57 (dq, J=14.6, 7.4 Hz, 1H), 0.92 (t, J=7.4 Hz, 3H). MS (ESI): [M+H]⁺=206.02.

### (1R,3S)-3-amino-4-(difluoromethylene)-1-ethylcyclopentane-1-carboxylic Acid ((R)-27b)

(R)-27b was synthesized from 25 and iodoethane following Method A to afford a white solid (29% for 2 steps). ¹H NMR (500 MHz, CD₃OD) δ 4.37 (m, 1H), 2.96-2.84 (m, 2H), 2.39 (dddd, J=15.4, 6.4, 4.4, 2.0 Hz, 1H), 1.91 (dq, J=14.9, 7.5 Hz, 1H), 1.73 (dq, J=14.8, 7.5 Hz, 1H), 1.58 (dd, J=13.1, 8.7 Hz, 1H), 0.94 (t, J=7.5 Hz, 3H). MS (ESI): [M+H]⁺=206.02.

### (1S,3S)-3-amino-4-(difluoromethylene)-1-propylcyclopentane-1-carboxylic Acid ((S)-27c)

(S)-27c was synthesized from 25 and 1-iodopropane following Method A to afford a colorless sticky liquid (16% for 2 steps). ¹H NMR (500 MHz, CD₃OD) δ 4.48-4.41 (m, 1H), 2.90 (dt, J=15.8, 4.5 Hz, 1H), 2.49 (dt, J=16.3, 3.1 Hz, 1H), 2.36-2.21 (m, 2H), 1.78 (td, J=12.8, 4.5 Hz, 1H), 1.55-1.45 (m, 1H), 1.42-1.17 (m, 2H), 0.94 (t, J=7.2 Hz, 3H). MS (ESI): [M+H]⁺=220.01.

### (1R,3S)-3-amino-4-(difluoromethylene)-1-propylcyclopentane-1-carboxylic Acid ((R)-27c)

(R)-27c was synthesized from 25 and 1-iodopropane following Method A to afford a colorless sticky liquid (22% for 2 steps). ¹H NMR (500 MHz, CD₃OD) δ 4.40-4.31 (m, 1H), 2.96-2.84 (m, 2H), 2.45-2.37 (m, 1H), 1.84 (ddd, J=12.9, 11.4, 5.5 Hz, 1H), 1.71-1.54 (m, 2H), 1.40-1.27 (m, 2H), 0.95 (t, J=6.7 Hz, 3H). MS (ESI): [M+H]⁺=220.01.

### (1S,3S)-3-amino-4-(difluoromethylene)-1-isobutylcyclopentane-1-carboxylic Acid ((S)-27d)

(S)-27d was synthesized from 25 and 1-iodo-2-methylpropane following Method A to afford a colorless sticky liquid (17% for 2 steps). ¹H NMR (500 MHz, CD₃OD) δ 4.47-4.41 (m, 1H), 2.92 (dt, J=15.8, 3.3 Hz, 1H), 2.48 (dt, J=16.2, 3.2 Hz, 1H), 2.39 (dd, J=14.3, 4.0 Hz, 1H), 2.26 (dd, J=14.3, 8.2 Hz, 1H), 1.82 (dd, J=14.0, 7.0 Hz, 1H), 1.68 (dp, J=13.3, 6.6 Hz, 1H), 1.49 (dd, J=13.9, 6.0 Hz, 1H), 0.93 (dd, J=12.2, 6.7 Hz, 6H). MS (ESI): [M+H]⁺=234.05.

### (1R,3S)-3-amino-4-(difluoromethylene)-1-isobutylcyclopentane-1-carboxylic Acid ((R)-27d)

(R)-27d was synthesized from 25 and 1-iodo-2-methylpropane following Method A to afford a yellow solid (40% for 2 steps). ¹H NMR (500 MHz, CD₃OD) δ 4.36-4.33 (m, 1H), 2.93 (d, J=13.7 Hz, 2H), 2.47-2.41 (m, 1H), 1.88-1.83 (m, 1H), 1.68-1.62 (m, 3H), 0.96-0.89 (m, 6H). MS (ESI): [M+H]⁺=234.05.

### (1S,3S)-3-amino-4-(difluoromethylene)-1-(fluoromethyl)cyclopentane-1-carboxylic Acid ((S)-27e)

(S)-27e was synthesized from 25 and fluoroiodomethane following Method B to afford a white solid (17% for 2 steps). ¹H NMR (500 MHz, CD₃OD) δ 4.61 (dd, J=46.8, 9.2 Hz, 1H), 4.55-4.40 (m, 2H), 2.92 (dtt, J=16.6, 4.0, 1.8 Hz, 1H), 2.64 (ddd, J=16.6, 3.4, 2.0 Hz, 1H), 2.49 (ddd, J=14.7, 8.2, 1.4 Hz, 1H), 2.38 (ddt, J=14.7, 5.0, 1.6 Hz, 1H). MS (ESI): [M+H]⁺=210.01.

### (1R,3S)-3-amino-4-(difluoromethylene)-1-(fluoromethyl)cyclopentane-1-carboxylic Acid ((R)-27e)

(R)-27e was synthesized from 25 and fluoroiodomethane following Method B to afford a white solid (15% for 2 steps). ¹H NMR (500 MHz, CD₃OD) δ 4.65 (ddd, J=49.0, 46.9, 9.2 Hz, 2H), 4.54-4.48 (m, 1H), 2.88-2.76 (m, 2H), 2.67 (dddd, J=16.0, 6.0, 4.5, 2.0 Hz, 1H), 1.93 (dd, J=13.8, 8.2 Hz, 1H). MS (ESI): [M+H]⁺=210.01.

### (1R,3S)-3-amino-4-(difluoromethylene)-1-(chloromethyl)cyclopentane-1-carboxylic Acid ((R)-27j)

(R)-27f was synthesized from 25 and chloroiodomethane following Method B to afford a colorless sticky liquid (18% for 2 steps). ¹H NMR (500 MHz, CD₃OD) δ 4.47-4.42 (m, 1H), 3.94 (d, J=10.9 Hz, 1H), 3.71 (d, J=10.9 Hz, 1H), 2.80 (dt, J=16.8, 3.5 Hz, 1H), 2.62 (dt, J=16.8, 3.8 Hz, 1H), 2.34 (d, J=5.5 Hz, 2H). MS (ESI): [M+H]⁺=225.95.

### (1S,3S)-3-amino-4-(difluoromethylene)-1-(hydroxymethyl)cyclopentane-1-carboxylic Acid ((S)-29)

(S)-29 was synthesized from 25 and iodomethyl pivalate following Method B to afford a colorless sticky liquid (12% for 2 steps). ¹H NMR (500 MHz, CD₃OD) δ 4.44-4.38 (m, 1H), 3.68-3.59 (m, 2H), 2.73 (ddd, J=16.5, 4.8, 2.1 Hz, 1H), 2.55 (dt, J=16.8, 3.8 Hz, 1H), 2.18 (dd, J=4.7, 1.4 Hz, 2H). MS (ESI): [M+H]⁺=208.04.

### (1R,3S)-3-amino-4-(difluoromethylene)-1-(hydroxymethyl)cyclopentane-1-carboxylic Acid ((R)-29)

(R)-29 was synthesized from 25 and iodomethyl pivalate following Method B to afford a colorless sticky liquid (31% for 2 steps). ¹H NMR (500 MHz, CD₃OD) δ 4.48-4.41 (m, 1H), 3.83-3.72 (m, 2H), 2.89-2.75 (m, 2H), 2.62 (dddd, J=16.2, 5.6, 4.2, 1.7 Hz, 1H), 1.92 (dd, J=14.1, 6.1 Hz, 1H). MS (ESI): [M+H]⁺=208.04.

### Ethyl (1S,3S)-3-((tert-butoxycarbonyl)amino)-4-(difluoromethylene)-1-formylcyclopentane-1-carboxylate (31)

To a solution of H₂SO₄ (0.22 mL, 4.1 mmol) in dry ethanol (4 mL) was added 29 (170 mg, 0.82 mmol). After stirring at 60 °C for 2 h, triethylamine (0.80 mL, 5.7 mmol) and di-tert-butyl dicarbonate (0.23 mL, 0.98 mmol) were added in sequence. Then the resulting solution was stirred for an additional 1 h at room temperature. The reaction mixture was concentrated, dissolved in ethyl acetate, and washed with 2 N HCl (aq.), saturated NaHCO₃ (aq.), and brine. The organic layer was dried over anhydrous Na₂SO₄, filtered, and concentrated in vacuo to afford an intermediate (ethyl (1S,3S)-3-((tert-butoxycarbonyl)amino)-4-(difluoromethylene)-1-(hydroxymethyl)cyclopentane-1-carboxylate) as a colorless sticky liquid (250 mg, 90%). To a solution of the intermediate (250 mg, 0.75 mmol) in DCM (5 mL) was added Dess-Martin periodinane (1.6 g, 3.7 mmol) and NaHCO₃ (310 mg, 3.7 mmol) at 0 °C. After stirring at 0 °C for 1 h, the reaction mixture was warmed to room temperature and stirred for 1 h. The reaction was quenched by adding saturated Na₂S₂O₃ (aq., 3 mL) and NaHCO₃ (aq., 9 mL) and stirred for a further 45 min. The layers were separated and the aqueous layer was extracted with DCM 2 times. The organic layers were combined, washed with brine, dried over anhydrous Na₂SO₄, filtered, and concentrated in vacuo to give a crude product. The crude product was further purified by silica gel column chromatography with 20% ethyl acetate in hexanes eluent to afford 31 as a colorless liquid (240 mg, 96%). ¹H NMR (500 MHz, CDCl₃) δ 9.66 (s, 1H), 4.85 (s, 1H), 4.73 (s, 1H), 4.27 (qd, J=7.1, 2.3 Hz, 2H), 2.98-2.89 (m, 1H), 2.85 (dt, J=16.6, 3.1 Hz, 1H), 2.55 (dd, J=13.8, 7.5 Hz, 1H), 2.26 (dd, J=13.7, 5.1 Hz, 1H), 1.44 (s, 9H), 1.31 (t, J=7.1 Hz, 3H). MS (ESI): [M+H]⁺=234.05.

### (1S,3S)-3-amino-4-(difluoromethylene)-1-vinylcyclopentane-1-carboxylic Acid (33)

KO^{t}Bu (200 mg, 1.8 mmol) and methyltriphenylphosphonium bromide (640 mg, 0.18 mmol) were dissolved into anhydrous THF under N₂ atmosphere and cooled to 0 °C. After stirring at 0 °C for 30 min, a solution of 31 (60 mg, 0.18 mmol) in anhydrous THF (2 mL) was added to the reaction mixture and stirred at room temperature for 30 min. The reaction mixture was washed with saturated NaHCO₃ (aq.) and extracted with ethyl acetate 2 times. The organic layers were then combined, washed with brine, dried over anhydrous Na₂SO₄, filtered, and concentrated in vacuo to give a crude product. The crude product was further purified by silica gel column chromatography with 7.5% ethyl acetate in hexanes eluent to afford an intermediate (ethyl (1S,3S)-3-((tert-butoxycarbonyl)amino)-4-(difluoromethylene)-1-vinylcyclopentane-1-carboxylate) as a colorless liquid (36 mg, 60%). The intermediate (6.0 mg, 0.018 mmol) was dissolved in 4 N HCl (aq.)/1,4-dioxane (10:1) mixture (0.5 mL) and the resulting solution was stirred at 60 °C for 20 h. The reaction mixture was concentrated in vacuo and washed with acetonitrile to afford 33 as a white solid (3.8 mg, 88%). ¹H NMR (500 MHz, CD₃OD) δ 6.02 (dd, J=17.4, 10.7 Hz, 1H), 5.27-5.19 (m, 2H), 4.40-4.32 (m, 1H), 3.00-2.91 (m, 1H), 2.75-2.67 (m, 1H), 2.55 (ddt, J=13.9, 7.9, 1.7 Hz, 1H), 2.30 (dd, J=13.9, 6.9 Hz, 1H). MS (ESI): [M+H]⁺=204.01.

### (1S,3S)-3-amino-4-(difluoromethylene)-1-ethynylcyclopentane-1-carboxylic Acid (35)

To a solution of 31 (50 mg, 0.15 mmol) in dry methanol (1 mL) was added K₂CO₃ (104 mg, 0.75 mmol) and dimethyl(1-diazo-2-oxopropyl)phosphonate (72 mg, 0.38 mmol) at 0 °C under N₂ atmosphere. After stirring at 0 °C for 1 h, the reaction was quenched by adding saturated NH₄Cl (aq.) at 0 °C and extracted with DCM 2 times. The organic layers were then combined, washed with brine, dried over anhydrous Na₂SO₄, filtered, and concentrated in vacuo to give a crude product. The crude product was further purified by silica gel column chromatography with 7.5% ethyl acetate in hexanes eluent to afford an intermediate (methyl (1S,3S)-3-((tert-butoxycarbonyl)amino)-4-(difluoromethylene)-1-ethynylcyclopentane-1-carboxylate) as a colorless liquid (33 mg, 70%). The intermediate (10 mg, 0.032 mmol) was dissolved in 4 N HCl (aq.)/1,4-dioxane (10:1) mixture (1 mL) and the resulting solution was stirred at 60 °C for 20 h. The reaction mixture was concentrated in vacuo and washed with acetonitrile to afford 35 as a white solid (6.2 mg, 82%). ¹H NMR (500 MHz, CD₃OD) δ 4.59-4.51 (m, 1H), 3.03 (dtd, J=15.5, 4.8, 1.8 Hz, 1H), 2.88 (s, 1H), 2.88-2.82 (m, 1H), 2.66 (ddt, J=13.5, 7.9, 1.8 Hz, 1H), 2.37 (dd, J=13.5, 7.3 Hz, 1H). MS (ESI): [M+H]⁺=201.99.

### Enzyme and Assays

GABA-AT, succinic semialdehyde dehydrogenase (SSDH)²¹, OAT²², and pyrroline-5-carboxylate reductase 1 (PYCR1)²³ were prepared by the previously described procedures. Enzyme assays for GABA-AT¹⁰ and OAT²⁴ were conducted according to the previously reported method with pH 8.5 and 8.0, respectively. The kinetic profiles were determined by the reported progress curve analysis²⁵ method.

### ¹F-NMR Studies

After complete inactivation by 27, the hOAT sample was washed with D₂O using Amicon® Ultra centrifugal filter (MilliporeSigma; Burlington, MA, USA) and diluted to ~20 μM with trifluoroethanol as an internal standard. The sample was run on Bruker Neo 600 MHz system with a QCI (HFCN) Cryo Probe w/ Z-Gradient at 298 K for 960 scans. For the denatured sample, the measured sample was heated to 55 °C with 10 equivalents of formic acid and measured with the same conditions.

### Fluoride Ion Release

The fluoride ion release assay was conducted according to the previously reported protocol²². The calculation for the proportion of PMP turnover was deducted under the assumption that every inactivation and turnover pathway is a first-order reaction. When the rate of inactivation is k₄ and the rate of turnover is k₃ with α-KG and the proportion of PMP turnover is x, the rate of inactivation under the absence of α-KG is k₄+xk₃, given that PMP turnover acts as inactivation. The rate of turnover without α-KG is then (1−x)k₃. Thus, a partition coefficient with α-KG (P₁) and a partition coefficient without α-KG (P₂) is presented as k₃/k₄ and k₃ (1−x)/(k₄+xk₃), respectively. Therefore, x can be calculated as (P₁−P₂)/P₁(1+P₂). For example, with 100% PMP turnover, P₂ is 0, which indicates that every pathway inactivates the enzyme without α-KG. Similar to this, with 100% PLP turnover, P₂ is the same as P₁, which indicates that the partition coefficient cannot be changed with or without α-KG.

### In Silico Construction of Intermediate Complexes

To build initial models for the external aldimine complex of CPP-115 in GABA-AT and OAT, molecular docking was conducted using Glide XP²⁶ in Schrödinger Release 2023-4 (Schrödinger; New York, NY, USA) package. The crystal structures of GABA-AT (PDB ID: 4YOD)¹⁰ and OAT (PDB ID: 7TED)¹⁵ were retrieved from the RCSB Protein Data Bank²⁷ (https://www.rcsb.org). The structures were prepared using Protein Prepare Wizard²⁸ in Schrödinger. Then, each input complex was solvated with the simple point charge (SPC) model of water box and neutralization by 0.15 M NaCl and minimized by Desmond²⁹ for 100 ps to relieve possible clashes and bad interactions to give the receptor input structure for docking. The receptor grid was generated, and the original ligand was removed by picking the ligand at the binding site. The appropriate external aldimine intermediate was generated using ChemDraw 22.2 (PerkinElmer; Waltham, MA, USA) and used for subsequent docking to the generated grid with van der Waals radii scaling factor of 0.80 to soften the potential. The resulting complex was then solvated, minimized, and MD-simulated with Desmond to generate natural structures with stable energy. The most stable complex was selected for each aminotransferase to analyze the predicted binding mode of the CPP-115 external aldimine intermediate complex. All structures in this study were visualized using the BIOVIA Discovery Studio 2022 visualizer (Dassault Systémes; San Diego, CA, USA).

### In Silico Evaluation of Compounds

To evaluate newly designed compounds, the corresponding external aldimine intermediates were prepared by the aforementioned method. The binding mode of each intermediate was then predicted by Glide XP, and the predicted binding mode was rescored using Prime MM-GBSA in Schrödinger.

### REFERENCES

(1) Roberts, E.; Frankel, S. y-Aminobutyric acid in brain: its formation from glutamic acid. J. Biol. Chem. 1950, 187, 55-63.
(2) Koh, W.; Kwak, H.; Cheong, E.; Lee, C. J. GABA tone regulation and its cognitive functions in the brain. Nat. Rev. Neurosci. 2023, 24 (9), 523-539.
(3) McQuail, J. A.; Frazier, C. J.; Bizon, J. L. Molecular aspects of age-related cognitive decline: the role of GABA signaling. Trends Mol. Med. 2015, 21 (7), 450-460.
(4) Hearl, W. G.; Churchich, J. E. Interactions between 4-aminobutyrate aminotransferase and succinic semialdehyde dehydrogenase, two mitochondrial enzymes. J. Biol. Chem. 1984, 259 (18), 11459-11463.
(5) James Willmore, L.; Abelson, M. B.; Ben-Menachem, E.; Pellock, J. M.; Donald Shields, W. Vigabatrin: 2008 update. Epilepsia 2009, 50 (2), 163-173.
(6) Juncosa, J. I.; Takaya, K.; Le, H. V.; Moschitto, M. J.; Weerawarna, P. M.; Mascarenhas, R.; Liu, D.; Dewey, S. L.; Silverman, R. B. Design and mechanism of (S)-3-amino-4-(difluoromethylenyl)cyclopent-1-ene-1-carboxylic acid, a highly potent γ-aminobutyric acid aminotransferase inactivator for the treatment of addiction. J. Am. Chem. Soc. 2018, 140 (6), 2151-2164.
(7) Lippert, B.; Metcalf, B. W.; Jung, M. J.; Casara, P. 4-amino-hex-5-enoic acid, a selective catalytic inhibitor of 4-aminobutyric-acid aminotransferase in mammalian brain. Eur. J. Biochem. 1977, 74 (3), 441-445.
(8) Nanavati, S. M.; Silverman, R. B. Mechanisms of inactivation of. gamma.-aminobutyric acid aminotransferase by the antiepilepsy drug 7-vinyl GABA (vigabatrin). J. Am. Chem. Soc. 1991, 113 (24), 9341-9349.
(9) Pan, Y.; Qiu, J.; Silverman, R. B. Design, synthesis, and biological activity of a difluoro-substituted, conformationally rigid vigabatrin analogue as a potent γ-aminobutyric acid aminotransferase inhibitor. J. Med. Chem. 2003, 46 (25), 5292-5293.
(10) Lee, H.; Doud, E. H.; Wu, R.; Sanishvili, R.; Juncosa, J. I.; Liu, D.; Kelleher, N. L.; Silverman, R. B. Mechanism of inactivation of γ-aminobutyric acid aminotransferase by (1S, 3S)-3-amino-4-difluoromethylene-1-cyclopentanoic acid (CPP-115). J. Am. Chem. Soc. 2015, 137(7), 2628-2640.
(11) Hwang, B.-Y.; Cho, B.-K.; Yun, H.; Koteshwar, K.; Kim, B.-G. Revisit of aminotransferase in the genomic era and its application to biocatalysis. J. Mol. Catal. B Enzym. 2005, 37 (1-6), 47-55.
(12) Peraino, C.; Bunville, L. G.; Tahmisian, T. N. Chemical, physical, and morphological properties of ornithine aminotransferase from rat liver. J. Biol. Chem. 1969, 244 (9), 2241-2249.
(13) Zigmond, E.; Ya'acov, A. B.; Lee, H.; Lichtenstein, Y.; Shalev, Z.; Smith, Y.; Zolotarov, L.; Ziv, E.; Kalman, R.; Le, H. V.; et al. Suppression of hepatocellular carcinoma by inhibition of overexpressed ornithine aminotransferase. ACS Med. Chem. Lett. 2015, 6 (8), 840-844.
(14) Zhu, W.; Doubleday, P. F.; Butrin, A.; Weerawarna, P. M.; Melani, R. D.; Catlin, D. S.; Dwight, T. A.; Liu, D.; Kelleher, N. L.; Silverman, R. B. Remarkable and unexpected mechanism for (S)-3-amino-4-(difluoromethylenyl) cyclohex-1-ene-1-carboxylic acid as a selective inactivator of human ornithine aminotransferase. J. Am. Chem. Soc. 2021, 143 (21), 8193-8207.
(15) Zhu, W.; Butrin, A.; Melani, R. D.; Doubleday, P. F.; Ferreira, G. M.; Tavares, M. T.; Habeeb Mohammad, T. S.; Beaupre, B. A.; Kelleher, N. L.; Moran, G. R. Rational Design, Synthesis, and Mechanism of (3 S, 4 R)-3-Amino-4-(difluoromethyl) cyclopent-1-ene-1-carboxylic Acid: Employing a Second-Deprotonation Strategy for Selectivity of Human Ornithine Aminotransferase over GABA Aminotransferase. J. Am. Chem. Soc. 2022, 144 (12), 5629-5642.
(16) Devitt, A. N.; Vargas, A. L.; Zhu, W.; Des Soye, B. J.; Butun, F. A.; Alt, T.; Kaley, N.; Ferreira, G. M.; Moran, G.; Kelleher, N. L. Design, Synthesis, and Mechanistic Studies of (R)-3-Amino-5, 5-difluorocyclohex-1-ene-1-carboxylic Acid as an Inactivator of Human Ornithine Aminotransferase. ACS Chem. Biol. 2024.
(17) Habeck, T.; Brown, K. A.; Des Soye, B.; Lantz, C.; Zhou, M.; Alam, N.; Hossain, M.

A.; Jung, W.; Keener, J. E.; Volny, M. Top-down mass spectrometry of native proteoforms and their complexes: a community study. Nat. Methods 2024, 1-9.
(18) Lee, H.; Juncosa, J. I.; Silverman, R. B. Ornithine aminotransferase versus GABA aminotransferase: implications for the design of new anticancer drugs. Med. Res. Rev. 2015, 35 (2), 286-305.
(19) Ruscio, J. Z.; Kohn, J. E.; Ball, K. A.; Head-Gordon, T. The influence of protein dynamics on the success of computational enzyme design. J. Am. Chem. Soc. 2009, 131 (39), 14111-14115.
(20) Weerawarna, P. M.; Moschitto, M. J.; Silverman, R. B. Theoretical and mechanistic validation of global kinetic parameters of the inactivation of GABA aminotransferase by OV329 and CPP-115. ACS Chem. Biol. 2021, 16 (4), 615-630.
(21) Silverman, R. B.; Bichler, K. A.; Leon, A. J. Unusual mechanistic difference in the inactivation of γ-aminobutyric acid aminotransferase by (E)- and (Z)-4-amino-6-fluoro-5-hexenoic acid. J. Am. Chem. Soc. 1996, 118 (6), 1253-1261.
(22) Mascarenhas, R.; Le, H. V.; Clevenger, K. D.; Lehrer, H. J.; Ringe, D.; Kelleher, N. L.; Silverman, R. B.; Liu, D. Selective targeting by a mechanism-based inactivator against pyridoxal 5′-phosphate-dependent enzymes: Mechanisms of inactivation and alternative turnover. Biochemistry 2017, 56 (37), 4951-4961.
(23) Christensen, E. M.; Patel, S. M.; Korasick, D. A.; Campbell, A. C.; Krause, K. L.; Becker, D. F.; Tanner, J. J. Resolving the cofactor-binding site in the proline biosynthetic enzyme human pyrroline-5-carboxylate reductase 1. J. Biol. Chem. 2017, 292 (17), 7233-7243.
(24) Juncosa, J. I.; Lee, H.; Silverman, R. B. Two continuous coupled assays for ornithine-δ-aminotransferase. Anal. Biochem. 2013, 440 (2), 145-149.
(25) Salminen, K. A.; Leppinen, J.; VenEllinen, J. I.; Pasanen, M.; Auriola, S.; Juvonen, R. O.; Raunio, H. Simple, direct, and informative method for the assessment of CYP2C19 enzyme inactivation kinetics. Drug Metab. Disposition 2011, 39 (3), 412-418.
(26) Friesner, R. A.; Murphy, R. B.; Repasky, M. P.; Frye, L. L.; Greenwood, J. R.; Halgren, T. A.; Sanschagrin, P. C.; Mainz, D. T. Extra precision glide: Docking and scoring incorporating a model of hydrophobic enclosure for protein-ligand complexes. J. Med. Chem. 2006, 49 (21), 6177-6196.
(27) Berman, H. M.; Westbrook, J.; Feng, Z.; Gilliland, G.; Bhat, T. N.; Weissig, H.; Shindyalov, I. N.; Bourne, P. E. The protein data bank. Nucleic Acids Res. 2000, 28 (1), 235-242.
(28) Madhavi Sastry, G.; Adzhigirey, M.; Day, T.; Annabhimoju, R.; Sherman, W. Protein and ligand preparation: parameters, protocols, and influence on virtual screening enrichments. J. Comput.-Aided Mol. Des. 2013, 27, 221-234.
(29) Bowers, K. J.; Chow, E.; Xu, H.; Dror, R. O.; Eastwood, M. P.; Gregersen, B. A.; Klepeis, J. L.; Kolossvary, I.; Moraes, M. A.; Sacerdoti, F. D. Scalable algorithms for molecular dynamics simulations on commodity clusters. In Proceedings of the 2006 ACM/IEEE Conference on Supercomputing, 2006; pp 84-es.
(30) Storici, P.; De Biase, D.; Bossa, F.; Bruno, S.; Mozzarelli, A.; Peneff, C.; Silverman, R. B.; Schirmer, T. Structures of γ-aminobutyric acid (GABA) aminotransferase, a pyridoxal 5′-phosphate, and [2Fe-2S] cluster-containing enzyme, complexed with γ-ethynyl-GABA and with the antiepilepsy drug vigabatrin. J. Biol. Chem. 2004, 279 (1), 363-373.
(31) Shen, B. W.; Hennig, M.; Hohenester, E.; Jansonius, J. N.; Schirmer, T. Crystal structure of human recombinant ornithine aminotransferase. J. Mol. Biol. 1998, 277 (1), 81-102.

What is claimed is:

1. A compound of formula (I), or a salt or hydrate thereof, 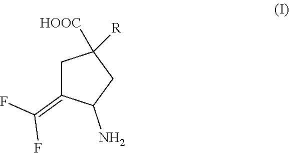
wherein
R is C₁-C₆-alkyl, C₂-C₆-alkenyl, or C₂-C₆-alkynyl, wherein the C₁-C₆-alkyl is optionally substituted with halogen or hydroxyl.

2. The compound of claim 1 having formula (Ia-A) or (Ia-B), or a salt or hydrate thereof,

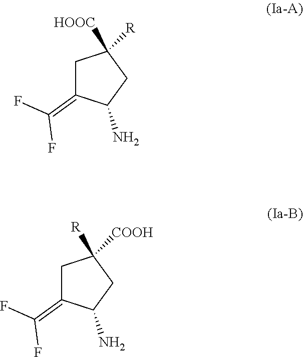

3. The compound of claim 1, wherein R is C₁-C₆-alkyl optionally substituted with halogen or hydroxyl.

4. The compound of claim 3, wherein R is methyl, ethyl, n-propyl, i-butyl, —CH₂F, —CH₂Cl, or —CH₂OH.

5. The compound of claim 1, wherein R is —CH═CH₂ or —C≡CH.

6. The compound of claim 1, wherein the compound is 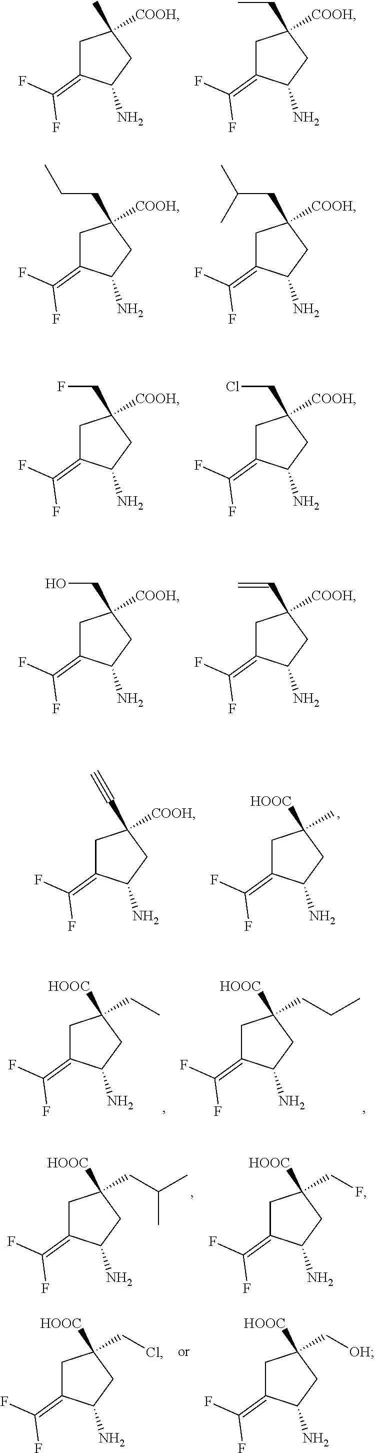
or a salt or hydrate thereof.

7. The compound of claim 1, wherein the compound is 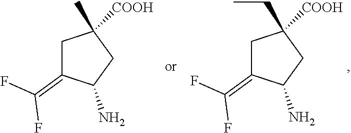
or a salt or hydrate thereof.

8. The compound of claim 1, wherein the compound has a selectivity for GABA-AT versus OAT of at least 20.

9. A salt of the compound of claim 1, having a formula of (Ib-A) or (Ib-B)

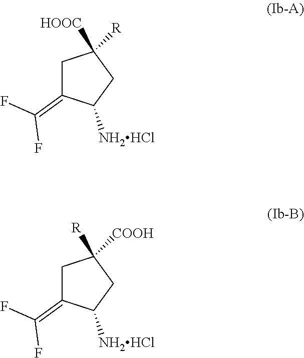

10. A pharmaceutical composition comprising the compound of claim 1 and a pharmaceutically acceptable carrier, diluent, or excipient.

11. A method of modulating γ-aminobutyric acid aminotransferase (GABA-AT) activity, the method comprising contacting the compound of claim 1, or a salt or hydrate thereof, with a medium comprising a GABA-AT, wherein the compound or the composition is present in an amount sufficient to modulate GABA-AT activity.

12. The method of claim 11, wherein the contact is in vivo.

13. A method for treating a disease or disorder associated with GABA-AT activity in a subject in need thereof, the method comprising administering to the subject a therapeutically effective amount of the compound of claim 1, or a salt or hydrate thereof.

14. The method of claim 13, wherein the disease or disorder is a neurological disease or disorder, or pain.

15. The method of claim 13, wherein the disease or disorder is selected from the group consisting of epilepsy, neuropathic pain, inflammatory pain, Alzheimer's disease, Parkinson's disease, neuropathic pain, partial seizures, complex partial seizures, secondary generalized seizures, tonic-clonic seizures, succinic semialdehyde dehydrogenase deficiency (SSADHD), infantile spasms in West's syndrome, Lennox-Gastaut syndrome, tubulous sclerosis, Tourette's syndrome, movement disorders, fibromyalgia, migraine related to epilepsy, restless leg syndrome and post-traumatic stress disorder, addiction, obesity, obsessive-compulsive disorders.

16. The method of claim 13, wherein the disease or disorder is selected from the group consisting of epilepsy, neuropathic pain, and inflammatory pain.

17. The compound of claim 2, wherein R is C₁-C₆-alkyl optionally substituted with halogen or hydroxyl.

18. The compound of claim 17, wherein R is methyl, ethyl, n-propyl, i-butyl, —CH₂F, —CH₂Cl, or —CH₂OH.

19. The compound of claim 2, wherein R is —CH═CH₂ or —C≡CH.

20. A method of modulating γ-aminobutyric acid aminotransferase (GABA-AT) activity, the method comprising contacting the pharmaceutical composition of claim 10 with a medium comprising a GABA-AT, wherein the compound or the composition is present in an amount sufficient to modulate GABA-AT activity.

21. A method for treating a disease or disorder associated with GABA-AT activity in a subject in need thereof, the method comprising administering to the subject a therapeutically effective amount of the pharmaceutical composition of claim 10.
